SEQUENCE LISTING (14 sequences)

SEQ ID NO: 1 - DNA (cDNA), 1668 bp; both-stranded, linear
  CDS 259..1491
ggatgatacaaatgtggaagatgcaaattgttcttcccctactttgatgagaaaaagtgcattgagtaaaatcatcttcaaaggacattaaacaataattccaaatcaccatcgccaactttttcaaatacaaatgcaacatctggctctccattgtcaaatctttctcgtgcaccattgagaaatttatctaatttcgttattccttcgtcagttaaatcaaaaacgaaacaatttacaaactctttgactcgttcaatgactgaagtggtttcgaaatcatcacactcatttttcaataatttgcatcttgcaacctcaactgcgtcttcttcagtatcgagcacaactcccaaaatagaattcaattccatagctgaaaatgatgatatccctaccaattatgactctgatgaagaattcgaagatggtgatacgtttatacaatccaccttgattcaccagttcaacgcaagtcaagtaacaacaacaacaataataataataccaatgatggtaacgacaataatatacctacaaaaattagacggttccactccatgtaccaaaccgataaagagattgcatcgtaccaacttcatgaagataattcattttgaaatttacaatattgaatattctcatctggagagtgatttgttaccacgaatcgatgctcatcaattagccagaatattacgtggagaccacgatgaccaatttgatgaatttattatcattgattgtcgatttgagtatgaatttaatggtggccatattactagggcaatcaatatctccacccaggaagcacttcaagaaaagctctttcaatatcaagaaacagataccaaggacactgaaagcaagaagcgattgataattttccattgtgagttcagtatgttcagaggaccaatgatggccaaacatttaagaaagtgtgatagaatgtgcaactacgacaattatcctctattaacataccccgatattgcaattttggaaggaggctataagaatttctatgaaaattacccccaatggtgtgatcctcaaggatatgtcgagatgaagaatttacgacacaaaaaattatgtgaatccaacttggataaagttagaaaagataataaactaactagagcaaagtcttatcaatttggtattcaacaccgccgtggtggttccactggtggacttttcggcaactataattacaacgttatgaactcatcagatcaacaattttggagtagcagtacttccaacactgctcaccacagaagtagtagcagtagcgggttcattaataatatgcatagtggtgcttcgtcatatcaccataggtcacaatcgtttgtaactatcaataatgagaaaattatcaagcgacaaagatcgactcccaaagtcagcaactcaccaaccaagccacctcatcaactgtatctcctgataaacccattccgttggctaatattcatagattaactcgtgccaacactatttcatcagaccaaacattgtttagcaataagctggtatcttccccaatgatatctccacttgcagctagttttgaacaatcgtcgattggaataagttcttctgaattatcagtcaatactcaagattttcaaccaccgactacgtcctttaggaatt

SEQ ID NO: 2 - DNA (cDNA), 786 bp; both-stranded, linear
  CDS 208..513
aacttgtttacttatttgtttatataattgatagatatcaattactaatttacccttgttttttacttcctactattcaagactttatttcctcctgataatcattttgtttgattatcattttcgtcaattagttcttttttttcatttgtttccagagtttaggaagactaccattttacaattttcaattcaaatattttcccaatgactaaaccaagatttttaacaagatatagaaagagcaaaagtgttggaatttcagatatgatccattacagtcccagatacagtgatgattcatacgagtatagacatgtgatgttacccaagaatatgttgaaagcaattcctcacgattactttaatcaagaaacaggtactttgaggatattgacagaagaagaatggagagggttaggaatcacacaatctttgggttgggcccattacgaaactcatgctccagagcctcatatattattattcaagagacccttaaatcccgggcaataaaaagattaactatatttgaatactatagaatcggaatcggttttaaagttaacactggaattaaaacataaaaaggaaagaaatagcccattggtcacagtaatctgtttccaacaacccccctcctcagaaataggatagaaatgaattaacgatgaatttgtatacactatttataagccaatctcattgattgcatttcttatttgtatattattaaatacgtatatcgcgagaaactgtataaatactcttggtacctcgcatgtt

SEQ ID NO: 3 - DNA (cDNA), 1002 bp; both-stranded, linear
  CDS 43..993
tagaacacacacatcccaagccaagaccaacacttattgcaaatggtagagttatctgattatcaacgtcaagaaaaagtcggagaaggtacttatggggttgtttataaagcattagataccaagcacaataatagagttgttgcattaaagaaaattcgattagaatcagaagatgaaggtgtacctagtaccgccattagagaaatctcgttattaaaagaaatgaaagatgataatatcgttcgattatatgatattattcattcagattctcataaattatatttagtatttgaatttttggatttagatttaaagaaatatatggaaagtattcctcaaggagttggactaggggctaatatgataaaaagatttatgaatcaattaattcgaggtattaaacattgtcattctcatcgagttttacatcgtgatttaaaaccacaaaatttattgattgataaagaagggaatttaaaattagcagattttggattagctcgagcatttggagttccattaagagcatatactcatgaagttgtcactttatggtatcgagctcccgaaatcttgttaggagggaaacaatattccactggggtagatatgtggtctgttggatgtatatttgctgaaatgtgtaataggaaaccattatttcctggtgattcagaaattgatgaaattttccgaattttccgaattttaggaacacctaatgaagaaatttggcctgatgttaattatttaccagattttaaatcaagtttccctcaatggaaaaagaaacctttgagtgaagcagttccaagtttggatgctaatggaattgatcttttggatcaaatgttggtgtatgatccaagtagaagaataagtgctaaacgagctttaattcatccttattttaatgataatgatgatcgtgatcataacaattataatgaagataatattgggattgacaaacaccaaaacatgcaataaatcttg

SEQ ID NO: 4 - DNA (cDNA), 1752 bp; both-stranded, linear
  CDS 184..1659
gctattccccccttttccttttttttatagagaaacttattccaattactcatcgaacaagatcttactagacttgtagactcacgacacgataaattttaattcattaatcaaccaacgaaccagccaaaccaaaattaattcacatttatactcactgtttgtcattttcatctcatagtaatgccacaagtcactaaaactaataatgaaaatgagtttagacttactagatcaaaagtacagcatcaagagtcgataagtaccatcaaaaatacgaccatatccaattctcagcataaacaacaaacacaacaacaaatttcatcaccacctcaagtctctgtaacatcatctgaaggagtttcacatgtcaatacacgtcaatatttgggtgatgtttcaaatcaatacataacaaatgctaaaccaacaaataaaagaaaaccattgggtggagacaatgcccctctacaaaaacaacagcatagaccatctagaccaatacccattgccagtgataacaacaataatggtagtaccagtagcagtagcaacagtagcaacaacaataacaacgacgcaaatagactagcatctttggcagttccatctcgattaccccaaaaacgacaagctactgaatcgtcgacaaatttagtagagaaattaagagtaccacaaccagaagtaggggaaagaagtcagtcataccataagaaatcacgtttaattgattatgaatggcaggatttggatgaagaagataatgacgaccaattaatggttagtgaatatgttaacgaaatattttcgtactattacgaattagaaacacgaatgttacctgatccgcaatatcttttcaaacaaacattgttaaaaccaagaatgagatcgatattggttgattggcttgttgaaatgcatttaaaattcaagttattacctgaatcactttttttggcagtcaatgtaatggatagattcatgtctgttgaagtggttcaaatagataaattacaattattggctacagcagctttatttactgctgccaaaaatgaagaagtattttctcccctggttaaaaattatgcatatttcactgatggttcatatactccagaagaagtggtacaagcagaaaaatacatgcttaccattcttaactttgatttgaattaccccaatccaatgaatttcttgagaagaatttctaaagctgatgattatgatgtccaatcaagaacgctaggaaaatatcttttggaaatcactatagttgattacaaatttattggtatgagaccatctttatgttgtgccctggccatgtatttagcaagactaatattgggcaaattgccagtttggaatgggaatttgattcattatagtggaggttatagaatcagtgatatgagagaatgtatcgaattaatgtttcaatatcttattgctcctatagaacatgatgaatttttcaaaaaatatgccatgagaaaatttatgagagcaagtactctttgtcgaaattgggctaaaaaattccaagcatcaggaagagatttgtttgatgaacgattatcgacccataggctaacattagaagatgatgacgaagaagaagaaatagtggtagcagaagcagaagagtaaagttttgaggactattggatctaggttcttatctttacaatgcataaatgaggaaatgaaagaagatgaacatgagttatgtgcattacc

SEQ ID NO: 5 - DNA (cDNA), 1070 bp; both-stranded, both
  CDS 30..1058
atcaaatccatcagagaaccacatcaatcatgtctactgcagcagttgcaacgaaaccatctgtcacttcaaaaccagcaactaaacaagttctgaattacaccaaagaaaaaaaagtaggggaaggtacatatgctgttgtgtacttgggtaaacaaatctccaccaaacgtcaaattgccatcaaagaaatcaaaacaggattattcaaagatgggttggatatgtcagcattgagagaagtgaaatatttgcaagaattgaaacatcccaatgttattgaactagtagatgtattttcagcaacaaataatttaaatttggtattagaatttctaccttgcgatttggaagtgttgatcaaagataaatcgattgttttcaaatcagcagatatcaaatcatggcttttaatgacattacgtgggatacatcattgtcatcggaattttattttacatcgtgatttgaaaccaaataatttattattggcaccggatggacaattgaaaatagcggattttggtcttgcacgagctttggtaaatcctaatgaagatttatcatctaatgttgtcactagatggtatagagcccctgaattattatttggtgctcgacattacactggagcagttgatatctggtcaataggtataatatttgctgaattaatgcttcgaataccttatttgccaggtaaagatgacgttgatcaattagatgttacatttagagcttatgggacaccaacagagcaaatatggccaaatgtttccagtttgccaatgtataatgcacttcatgtgtatccacctccttcaagacaagaattacgtaatagatttagtgctgctacggaaaaagcccttgatttgttgatatcgatgacccaattggatccaagtagaagatgtgattctacactagcattattacacgattattttactgaatcgcctcgtcctactgacccgaaaaagttgcctaaaaagtcttctccagaaaagagagaaaatgaagatgaacagaataatggctctaaaagaaggcatgtttaggtttctata

SEQ ID NO: 6 - DNA (cDNA), 477 bp; both-stranded, linear
  CDS 1..477
tgttcagctattgatacgaaaagttcagtctcagcgatggagcacaagattgctataaagaaagtaacaaagattttcaacaaagacatccttctaatcagggcaatacgagagcttaagttcatgatgtttttcagaggccacaagaatattgcaactttgcttgacttagatgttgtatatgtgaagccttatgaaggcttgtattgttttcaagagctagccgatttagatttagctcgtgttttgtactcaaacgtccaattttcagaatttcacattcaaagctttatgtaccaaattctttgcggactcaagtacatccattctgctgatgtaatacatcgggacctaaagccaggaaacatattggtcaccactcaagggactttaaaaatatgtgatttcggcttagcacgaggaataaatcctgtatatttcagaaaccgctcagctgtcatcacaaactacgtagca

SEQ ID NO: 7 - protein, 411 aa
MTEVVSKSSHSFFNNLHLATSTASSSVSSTTPKIEFNSIAENDDIPTNYDSDEEFEDGDTFIQSTLIHQFNASQVTTTTIIIIPMMVTTIIYLQKLDGSTPCTKPIKRLHRTNFMKIIHFEIYNIEYSHLESDLLPRIDAHQLARILRGDHDDQFDEFIIIDCRFEYEFNGGHITRAINISTQEALQEKLFQYQETDTKDTESKKRLIIFHCEFSMFRGPMMAKHLRKCDRMCNYDNYPLLTYPDIAILEGGYKNFYENYPQWCDPQGYVEMKNLRHKKLCESNLDKVRKDNKLTRAKSYQFGIQHRRGGSTGGLFGNYNYNVMNSSDQQFWSSSTSNTAHHRSSSSSGFINNMHSGASSYHHRSQSFVTINNEKIIKRQRSTPKVSNSPTKPPHQLYLLINPFRWLIFID

SEQ ID NO: 8 - protein, 102 aa
MTKPRFLTRYRKSKSVGISDMIHYSPRYSDDSYEYRHVMLPKNMLKAIPHDYFNQETGTLRILTEEEWRGLGITQSLGWAHYETHAPEPHILLFKRPLNPGQ

SEQ ID NO: 9 - protein, 317 aa
MVELSDYQRQEKVGEGTYGVVYKALDTKHNNRVVALKKIRLESEDEGVPSTAIREISLLKEMKDDNIVRLYDIIHSDSHKLYLVFEFLDLDLKKYMESIPQGVGLGANMIKRFMNQLIRGIKHCHSHRVLHRDLKPQNLLIDKEGNLKLADFGLARAFGVPLRAYTHEVVTLWYRAPEILLGGKQYSTGVDMWSVGCIFAEMCNRKPLFPGDSEIDEIFRIFRILGTPNEEIWPDVNYLPDFKSSFPQWKKKPLSEAVPSLDANGIDLLDQMLVYDPSRRISAKRALIHPYFNDNDDRDHNNYNEDNIGIDKHQNMQ

SEQ ID NO: 10 - protein, 492 aa
MPQVTKTNNENEFRLTRSKVQHQESISTIKNTTISNSQHKQQTQQQISSPPQVSVTSSEGVSHVNTRQYLGDVSNQYITNAKPTNKRKPLGGDNAPLQKQQHRPSRPIPIASDNNNNGSTSSSSNSSNNNNNDANRLASLAVPSRLPQKRQATESSTNLVEKLRVPQPEVGERSQSYHKKSRLIDYEWQDLDEEDNDDQLMVSEYVNEIFSYYYELETRMLPDPQYLFKQTLLKPRMRSILVDWLVEMHLKFKLLPESLFLAVNVMDRFMSVEVVQIDKLQLLATAALFTAAKNEEVFSPLVKNYAYFTDGSYTPEEVVQAEKYMLTILNFDLNYPNPMNFLRRISKADDYDVQSRTLGKYLLEITIVDYKFIGMRPSLCCALAMYLARLILGKLPVWNGNLIHYSGGYRISDMRECIELMFQYLIAPIEHDEFFKKYAMRKFMRASTLCRNWAKKFQASGRDLFDERLSTHRLTLEDDDEEEEIVVAEAEE

SEQ ID NO: 11 - protein, 343 aa
MSTAAVATKPSVTSKPATKQVLNYTKEKKVGEGTYAVVYLGKQISTKRQIAIKEIKTGLFKDGLDMSALREVKYLQELKHPNVIELVDVFSATNNLNLVLEFLPCDLEVLIKDKSIVFKSADIKSWLLMTLRGIHHCHRNFILHRDLKPNNLLLAPDGQLKIADFGLARALVNPNEDLSSNVVTRWYRAPELLFGARHYTGAVDIWSIGIIFAELMLRIPYLPGKDDVDQLDVTFRAYGTPTEQIWPNVSSLPMYNALHVYPPPSRQELRNRFSAATEKALDLLISMTQLDPSRRCDSTLALLHDYFTESPRPTDPKKLPKKSSPEKRENEDEQNNGSKRRHV

SEQ ID NO: 12 - protein, 159 aa
CSAIDTKSSVSAMEHKIAIKKVTKIFNKDILLIRAIRELKFMMFFRGHKNIATLLDLDVVYVKPYEGLYCFQELADLDLARVLYSNVQFSEFHIQSFMYQILCGLKYIHSADVIHRDLKPGNILVTTQGTLKICDFGLARGINPVYFRNRSAVITNYVA

SEQ ID NO: 13 - DNA (cDNA), 1019 bp; both-stranded, linear
  CDS 1..1017
atgaagttgtcagattattatatagacaaggaattaatttacaatagtgccatttctgatatatatacggctattgataagtttaataacttaccagtatgtcttaaaatagttgatgaagatttcagtcttccaccacattcaatccatcgagaaatttttatacttaaaactttgaaaccacatccaaacataattgaatattttaatgatcttaaaatttatgatgatgttatattagtcaccaaattgtatcgttatgatttgagtcaattgattgaaattacaaaatattgtaaacgaacaacacgatttatttatggtattaatggtaatcttgttagtaatcaatatacacttgctaatgaaattgaagaaaaagatatcaaattatggttaaaatcaatgagttcaggacttgaatttattcattcacaagggataattcatcgtgatataaaacccagtaatattttctttgcccgggatgatataacacaaccgattattggagattttgatatttgttatgatttaaaactgccacctaaagatgaaccccctatggcgaaatatattgatgtatctacaggtatttataaagcaccagaattgattcttggtataactaattatgaatatgaaattgatatttggtcattgggtataattttgactggtttatattcagaaaattttcaaagtgttttagtcaaagatgataaagaattgactaatgattctcatgttagtgatttatatttattaaatcaaatatttgaaaatttcggtacacccaatttaactgattttgaagatgaattattttgtgatgaatataataatgaaaacttgcattttaaaaaattcaatttacaaaaatatcctagaaaagattgggatattattttacctcgatgcaatgatgatttaatgaaagaaatttttaccaagatgattagatatgatcgaagtaaaagaataacttctaaagaaatcttacaattaatgttggattg

SEQ ID NO: 14 - protein, 339 aa
MKLSDYYIDKELIYNSAISDIYTAIDKFNNLPVCLKIVDEDFSLPPHSIHREIFILKTLKPHPNIIEYFNDLKIYDDVILVTKLYRYDLSQLIEITKYCKRTTRFIYGINGNLVSNQYTLANEIEEKDIKLWLKSMSSGLEFIHSQGIIHRDIKPSNIFFARDDITQPIIGDFDICYDLKLPPKDEPPMAKYIDVSTGIYKAPELILGITNYEYEIDIWSLGIILTGLYSENFQSVLVKDDKELTNDSHVSDLYLLNQIFENFGTPNLTDFEDELFCDEYNNENLHFKKFNLQKYPRKDWDIILPRCNDDLMKEIFTKMIRYDRSKRITSKEILQLMLD

(19) United States
(12) Patent Application Publication
(10) Pub. No.: US 2003/0104362 A1
(43) Pub. Date: Jun. 5, 2003

(54) CELL-CYCLE REGULATORY PROTEINS FROM HUMAN PATHOGENS, AND USES RELATED THERETO

(76) Inventors: GUILLAUME COTTAREL, ARLINGTON, MA (US); VERONIQUE DAMAGNEZ, CAMBRIDGE, MA (US); GIULIO DRAETTA, OPERA (IT)

Correspondence Address: ROPES & GRAY, ONE INTERNATIONAL PLACE, BOSTON, MA, 02110-2624, US

(21) Appl. No.: 09/072,994

(22) Filed: May 5, 1998

Related U.S. Application Data
(63) Continuation-in-part of application No. 08/463,090, filed on Jun. 5, 1995, now Pat. No. 5,801,015.

(51) Int. Cl. C12Q 1/68; C07H 21/04; C12N 9/12; C12N 1/18; C12N 15/74

(52) U.S. Cl. 435/6; 435/69.1; 435/194; 435/254.22; 435/320.1; 536/23.2

(57) ABSTRACT

The present invention relates to the discovery of novel cell cycle regulatory proteins from the human pathogen Candida.

### RELATED APPLICATIONS

This application is a continuation-in-part of U.S. Ser. No. 08/463,090, filed Jun. 5, 1995, the contents of which are incorporated herein by reference.

### BACKGROUND OF THE INVENTION

The progression of a proliferating eukaryotic cell through the cell-cycle checkpoints is controlled by an array of regulatory proteins that guarantee that mitosis occurs at the appropriate time. Protein phosphorylation is the most common post-translational modification that regulates processes inside the cells, and a large number of cell cycle transitions are regulated by, in addition to protein-protein interactions, the phosphorylation states of various proteins. In particular, the execution of various stages of the cell-cycle is generally believed to be under the control of a large number of mutually antagonistic kinases and phosphatases. A paradigm for these controls is the CDC2 protein kinase, a cyclin-dependent kinase (CDK) whose activity is required for the triggering of mitosis in eukaryotic cells (for reviews, see Hunt (1989) Curr. Opin. Cell Biol. 1:268-274; Lewin (1990) Cell 61:743-752; and Nurse (1990) Nature 344:503-508). During mitosis, the CDC2 kinase appears to trigger a cascade of downstream mitotic phenomena such as metaphase alignment of chromosomes, segregation of sister chromatids in anaphase, and cleavage furrow formation. Many target proteins involved in mitotic entry of the proliferating cell are directly phosphorylated by the CDC2 kinase. For instance, the CDC2 protein kinase acts by phosphorylating a wide variety of mitotic substrates involved in regulating the cytoskeleton of cells, such that entry into mitosis is coordinated with dramatic rearrangement of cytoskeletal elements.

The CDC2 kinase is subject to multiple levels of control. One well-characterized mechanism regulating the activity of CDC2 involves the phosphorylation of tyrosine, threonine, and serine residues; the phosphorylation level of which varies during the cell-cycle (Krekk et al. (1991) EMBO J 10:305-316; Draetta et al. (1988) Nature 336:738-744; Dunphy et al. (1989) Cell 58:181-191; Morla et al. (1989) Cell 58:193-203; Gould et al. (1989) Nature 342:39-45; and Solomon et al. (1990) Cell 63:1013-1024). The phosphorylation of CDC2 on Tyr-15 and Thr-14, two residues located in the putative ATP binding site of the kinase, negatively regulates kinase activity. This inhibitory phosphorylation of CDC2 is mediated at least in part by the wee1 and mik1 tyrosine kinases (Russel et al. (1987) Cell 49:559-567; Lundgren et al. (1991) Cell 64:1111-1122; Featherstone et al. (1991) Nature 349:808-811; and Parker et al. (1992) PNAS 89:2917-2921). These kinases act as mitotic inhibitors, over-expression of which causes cells to arrest in the G2 phase of the cell-cycle. By contrast, loss of function of wee1 causes a modest advancement of mitosis, whereas loss of both wee1 and mik1 function causes grossly premature mitosis, uncoupled from all checkpoints that normally restrain cell division (Lundgren et al. (1991) Cell 64:1111-1122).

As the cell is about to reach the end of G2, dephosphorylation of the CDC2-inactivating Thr-14 and Tyr-15 residues occurs leading to activation of the CDC2 complex as a kinase. A stimulatory phosphatase, known as CDC25, is responsible for Tyr-15 and Thr-14 dephosphorylation and serves as a rate-limiting mitotic activator. (Dunphy et al. (1991) Cell 67:189-196; Lee et al. (1992) Mol. Biol. Cell. 3:73-84; Millar et al. (1991) EMBO J 10:4301-4309; and Russell et al. (1986) Cell 45:145-153). Recent evidence indicates that both the CDC25 phosphatase and the CDC2-specific tyrosine kinases are detectably active during interphase, suggesting that there is an ongoing competition between these two activities prior to mitosis (Kumagai et al. (1992) Cell 70:139-151; Smythe et al. (1992) Cell 68:787-797; and Solomon et al. (1990) Cell 63:1013-1024). This situation implies that the initial decision to enter mitosis involves a modulation of the equilibrium of the phosphorylation state of CDC2 which is likely controlled by variation of the rate of tyrosine dephosphorylation of CDC2 and/or a decrease in the rate of its tyrosine phosphorylation. A variety of genetic and biochemical data appear to favor a decrease in CDC2-specific tyrosine kinase activity near the initiation of mitosis which can serve as a triggering step to tip the balance in favor of CDC2 dephosphorylation (Smythe et al. (1992) Cell 68:787-797; Matsumoto et al. (1991) Cell 66:347-360; Kumagai et al. (1992) Cell 70:139-151; Rowley et al. (1992) Nature 356:353-355; and Enoch et al. (1992) Genes Dev. 6:2035-2046). Moreover, recent data suggests that the activated CDC2 kinase is responsible for phosphorylating and activating CDC25. This event would provide a self-amplifying loop and trigger a rapid increase in the activity of the CDC25 protein, ensuring that the tyrosine dephosphorylation of CDC2 proceeds rapidly to completion (Hoffmann et al. (1993) EMBO J. 12:53).

Although many fungal genera have been identified as etiologic opportunistic infections, it is known that Candida constitute the majority of the pathogens involved in these infections. Candida is unique among opportunistic pathogens because it is a resident fungus found in the normal flora of mucosa and skin of many animals, including humans. Although there are numerous species of Candida, the majority of infections are caused by C. albicans and C. tropicalis.

Clinical diagnosis and treatment of systemic fungemia suffers several shortcomings compared to bacterial septicemia. First, many of the approved antifungal therapeutics are more toxic to the patient than analogous antibacterial agents. As a result, clinicians desire a more reliable demonstration of fungemia before prescribing antifungal agents. Second, fungemic patients have a poor prognosis, unless diagnosed early in infection. Third, fungi generally grow slower than the major barceremic organisms, and consequently diagnosis requiring an in vitro culture step is time consuming. And fourth, some of the fungi (again in diagnosis requiring in vitro cultivation) will not yield colonies on synthetic media for weeks, if at all.

### SUMMARY OF THE INVENTION

The present invention provides reagents and assays which permit rapid detection and evaluation of Candida yeast infections without employing culturing, incubation, sub-culturing or microscopic examination.

The present invention also makes available reagents and assays for identifying compounds which have antifungal properties and which may be used as anti-mycotic agents. Such agents developed with the subject assays can be used therapeutically, as well as, for example, preservatives in foodstuff, feed supplement for promoting weight gain in livestock, or in disinfectant formulations for treatment of non-living matter, e.g., for decontaminating hospital equipment and rooms.

In particular, the present invention relates to the discovery of novel cell-cycle regulatory proteins from animal pathogens, particularly from members of the genus Candida. One aspect of the invention features a Candida TYP1 polypeptide, preferably a substantially pure preparation of a TYP1 polypeptide, or a recombinant TYP1 polypeptide. The TYP1 protein shares certain features which suggest that it is a homolog to the S. Pombe cdc25 phosphatase. In preferred embodiments, the biological activity of the polypeptide includes the ability to specifically bind a cyclin dependent kinase (CDK). Preferably, the TYP1 polypeptide has a phosphatase activity, e.g. a phosphotyrosine phosphatase activity, e.g. a phosphoserine/phosphothreonine phosphatase activity. The TYP1 polypeptide may also generally be characterized as having an amino acid sequence at least 60%, 80%, 90% or 95% homologous to the amino acid sequence in SEQ ID No: 7, in addition to those forms of the polypeptide which comprise an amino acid sequence identical to the polypeptide designated by SEQ ID No: 7. Preferred TYP1 polypeptides are at least 5, 10, 20, 50, 100, or 150 amino acids in length; e.g., the polypeptide comprises at least 5, preferably at least 10, more preferably at least 20, more preferably at least 50, 100, or 150 contiguous amino acids from SEQ ID No: 7. Moreover, the subject TYP1 polypeptides can either mimic (agonize) or inhibit (antagonize) the biological activity of the wild-type form of the phosphatase, e.g., of its ability to regulate Candida cell proliferation. In preferred embodiments, the TYP1 polypeptide is isolated or is a recombinant form of a gene expressed by one of Candida albicans, Candida stellatoidea, Candida tropicalis, Candida parapsilosis, Candida krusei, Candida pseudotropicalis, Candida quillermondii, Candida glabrata, Candida lusianiae, or Candida rugosa.

Another aspect of the invention features a Candida CKS1 polypeptide, preferably a substantially pure preparation of a CKS1 polypeptide, or a recombinant CKS1 polypeptide. In preferred embodiments, the biological activity of the polypeptide includes the ability to specifically bind a cyclin dependent kinase (CDK). Preferably, the CKS1 polypeptide modulates the kinase activity of a CDK. The CKS1 polypeptide may generally be characterized as having an amino acid sequence at least 60%, 80%, 90% or 95% homologous to the amino acid sequence in SEQ ID No: 8, in addition to those forms of the polypeptide which comprise an amino acid sequence identical to the polypeptide designated by SEQ ID No: 8. Preferred CKS1 polypeptides are at least 5, 10, 20, 50 or 75 amino acids in length; e.g., the polypeptide comprises at least 5, preferably at least 10, more preferably at least 20, more preferably at least 50 or 75 contiguous amino acids from SEQ ID No: 8. Moreover, the subject CKS1 polypeptides can either mimic (agonize) or inhibit (antagonize) the biological activity of the wild-type form of the protein, e.g., of its ability to regulate Candida cell proliferation. In preferred embodiments, the CKS1 polypeptide is isolated from one of Candida albicans, Candida stellatoidea, Candida tropicalis, Candida parapsilosis, Candida krusei, Candida pseudotropicalis, Candida quillermondii, Candida glabrata, Candida lusianiae, or Candida rugosa.

Another aspect of the invention features a Candida CDK1 polypeptide, preferably a substantially pure preparation of a CDK1 polypeptide, or a recombinant CDK1 polypeptide. In preferred embodiments, the biological activity of the polypeptide includes the ability to specifically bind a cyclin. Preferably, the CDK1 polypeptide has an intrinsic kinase activity, which may depend on formation of a complex with a cyclin. The CDK1 polypeptide may generally be characterized as having an amino acid sequence at least 60%, 80%, 90% or 95% homologous to the amino acid sequence in SEQ ID No: 9, in addition to those forms of the polypeptide which comprise an amino acid sequence identical to the polypeptide designated by SEQ ID No: 9. Preferred CDK1 polypeptides are at least 5, 10, 20, 50, 100 or 150 amino acids in length; e.g., the polypeptide comprises at least 5, preferably at least 10, more preferably at least 20, more preferably at least 50, 100 or 150 contiguous amino acids from SEQ ID No: 9. Moreover, the subject CDK1 polypeptides can either mimic (agonize) or inhibit (antagonize) the biological activity of the wild-type form of the kinase, e.g., of its ability to regulate Candida cell proliferation. In preferred embodiments, the CDK1 polypeptide is isolated from one of Candida albicans, Candida stellatoidea, Candida tropicalis, Candida parapsilosis, Candida krusei, Candida pseudotropicalis, Candida quillermondii, Candida glabrata, Candida lusianiae, or Candida rugosa.

Another aspect of the invention features a Candida CYB1 polypeptide, preferably a substantially pure preparation of a CYB1 polypeptide, or a recombinant CYB1 polypeptide. In preferred embodiments, the biological activity of the polypeptide includes the ability to specifically bind a cyclin dependent kinase (CDK). Preferably, the CYB1 polypeptide modulates the kinase activity of a CDK. The CYB1 polypeptide may generally be characterized as having an amino acid sequence at least 60%, 80%, 90% or 95% homologous to the amino acid sequence in SEQ ID No: 10, in addition to those forms of the polypeptide which comprise an amino acid sequence identical to the polypeptide designated by SEQ ID No: 10. Preferred CYB1 polypeptides are at least 5, 10, 20, 50, 100 or 150 amino acids in length; e.g., the polypeptide comprises at least 5, preferably at least 10, more preferably at least 20, more preferably at least 50, 100 or 150 contiguous amino acids from SEQ ID No: 10. Moreover, the subject CYB1 polypeptides can either mimic (agonize) or inhibit (antagonize) the biological activity of the wild-type form of the protein, e.g., of its ability to regulate Candida cell proliferation. In preferred embodiments, the CYB1 polypeptide is isolated from one of Candida albicans, Candida stellatoidea, Candida tropicalis, Candida parapsilosis, Candida krusei, Candida pseudotropicalis, Candida quillermondii, Candida glabrata, Candida lusianiae, or Candida rugosa.

Still another aspect of the invention features a Candida CAK1 polypeptide, preferably a substantially pure preparation of a CAK1 polypeptide, or a recombinant CAK1 polypeptide. In preferred embodiments, the biological activity of the polypeptide includes the ability to specifically bind a cyclin dependent kinase (CDK). Preferably, the CAK1 polypeptide has a kinase activity, e.g. a serine/threonine kinase activity. The CAK1 polypeptide may also generally be characterized as having an amino acid sequence at least 60%, 80%, 90% or 95% homologous to the amino acid sequence in SEQ ID No: 14, in addition to those forms of the polypeptide which comprise an amino acid sequence identical to the polypeptide designated by SEQ ID No: 14. Preferred CAK1 polypeptides are at least 5, 10, 20, 50, 100, or 150 amino acids in length; e.g., the polypeptide comprises at least 5, preferably at least 10, more preferably at least 20, more preferably at least 50, 100, or 150 contiguous amino acids from SEQ ID No: 14. Moreover, the subject CAK1 polypeptides can either mimic (agonize) or inhibit (antagonize) the biological activity of the wild-type form of the kinase, e.g., of its ability to regulate Candida cell proliferation. In preferred embodiments, the CAK1 polypeptide is isolated from one of Candida albicans, Candida stellatoidea, Candida tropicalis, Candida parapsilosis, Candida krusei, Candida pseudotropicalis, Candida quillermondii, Candida glabrata, Candida lusianiae, or Candida rugosa.

Another aspect of the invention features a Candida MOC1 polypeptide, preferably a substantially pure preparation of a MOC1 polypeptide, or a recombinant MOC1 polypeptide. In preferred embodiments, the biological activity of the polypeptide includes the ability to specifically bind a cyclin dependent kinase (CDK). Preferably, the MOC1 polypeptide has a kinase activity, e.g. a serine/threonine kinase activity. The MOC1 polypeptide may also generally be characterized as having an amino acid sequence at least 60%, 80%, 90% or 95% homologous to the amino acid sequence in SEQ ID No: 11, in addition to those forms of the polypeptide which comprise an amino acid sequence identical to the polypeptide designated by SEQ ID No: 11. Preferred MOC1 polypeptides are at least 5, 10, 20, 50, 100, or 150 amino acids in length; e.g., the polypeptide comprises at least 5, preferably at least 10, more preferably at least 20, more preferably at least 50, 100, or 150 contiguous amino acids from SEQ ID No: 1. Moreover, the subject MOC1 polypeptides can either mimic (agonize) or inhibit (antagonize) the biological activity of the wild-type form of the kinase, e.g., of its ability to regulate Candida cell proliferation. In preferred embodiments, the MOC1 polypeptide is isolated from one of Candida albicans, Candida stellatoidea, Candida tropicalis, Candida parapsilosis, Candida krusei, Candida pseudotropicalis, Candida quillermondii, Candida glabrata, Candida lusianiae, or Candida rugosa.

In yet other preferred embodiments, the subject regulatory proteins can be provided as recombinant fusion proteins which includes a second polypeptide portion, e.g., a second polypeptide having an amino acid sequence unrelated to a protein represented by one of SEQ ID Nos: 7-12 or 14, e.g. the second polypeptide portion is glutathione-S-transferase, e.g. the second polypeptide portion is a DNA binding domain, e.g. the second polypeptide portion is a polymerase activating domain, e.g. the fusion protein is functional in a two-hybrid assay.

Yet another aspect of the present invention concerns an immunogen comprising at least a portion of a polypeptide designated by one of SEQ ID Nos. 7-12 or 14 in an immunogenic preparation, the immunogen being capable of eliciting an immune response specific for the subject protein; e.g., a humoral response, e.g., an antibody response; e.g., a cellular response.

A still further aspect of the present invention features an antibody preparation specifically reactive with an epitope of one of the subject regulatory proteins.

Another aspect of the present invention provides a substantially pure nucleic acid having a nucleotide sequence which encodes one of the subject polypeptides. Furthermore, in certain preferred embodiments, the subject nucleic acids will include a transcriptional regulatory sequence, e.g. at least one of a transcriptional promoter or transcriptional enhancer sequence, operably linked to the recombinant gene sequence, e.g., to render the recombinant gene sequence suitable for use as an expression vector.

In yet a further preferred embodiment, the nucleic acid hybridizes under stringent conditions to a nucleic acid probe corresponding to at least 20 consecutive nucleotides of a gene designated by one of SEQ ID Nos: 1-6 or 13; more preferably it hybridizes to at least 40 consecutive nucleotides of one of SEQ ID Nos: 1-6 or 13; and even more preferably it hybridizes to at least 60, 90 or 120 consecutive nucleotides of one of SEQ ID Nos: 1-6 or 13.

In addition, the present invention makes available assays and reagents for identifying anti-proliferative agents, such as mitotic and meiotic inhibitors, which act by inhibiting biological action of one of the subject regulatory proteins. The subject assays include those designed to identify agents which disrupt binding to other regulatory proteins, as well as (if applicable) agents which function as inhibitors of the catalytic activity of the subject protein.

Other features and advantages of the invention will be apparent from the following detailed description, and from the claims. The practice of the present invention will employ, unless otherwise indicated, conventional techniques of cell biology, cell culture, molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press:1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986).

### DESCRIPTION OF THE DRAWINGS

FIG. 1A demonstrates a Lineweaver-Burke analysis for recombinant Candida TYP1 hydrolysis of para-nitrophenylphospate.

FIG. 1B demonstrates a Lineweaver-Burke analysis for recombinant Candida TYP1 hydrolysis of fluoroscein diphosphate.

### DETAILED DESCRIPTION OF THE INVENTION

Protein phosphorylation is the most common post-translational modification that regulates processes inside cells and plays a key role in regulating the cell cycle engine. Protein kinases add phosphates to proteins by transferring phosphate groups from, for example, ATP, to hydroxyl groups on amino acid side chains; protein phosphatases remove the phosphate group. Phosphorylation of a given amino acid in a protein can have a variety of effects: activating or inactivating a protein's enzymatic activity, or altering a protein's affinity for binding to other proteins. In dividing eukaryotic cells, circuits of regulatory kinases and phosphatases oversee both the initiation and completion of the major transitions of both the meiotic and mitotic cell-cycles. These regulatory networks guarantee that the successive events of each cell-cycle occur in a faithful and punctual manner. Passage of a cells through the cell cycle is regulated at a number of key control points. For example, mitosis cannot begin until the cell has grown sufficiently and replicated its genome accurately. Likewise, cell division cannot ensue until the mitotic spindle has distributed the chromosomes equally to both daughter cells.

In fission and budding yeasts, CDC2 (CDC28 in budding yeast) is the catalytic subunit of a protein kinase complex which is required for both DNA synthesis and for entry into mitosis. The timing and activation of the CDC2 kinase is regulated by a physical association with regulatory subunits called cyclins, as well as a network of protein kinases and phosphatases. For example, inhibitory phosphorylation of Tyr-15 and/or Thr-14 is mediated by the antagonistic actions of the Wee1 protein kinase and the CDC25 tyrosine phosphatase, the dephosphorylating activity of the latter resulting in activation a kinase activity of a CDC2/cyclin complex. Moreover, cyclins and cyclin dependent kinases (CDK), such as CDC2, are key components of the eukaryotic cell cycle in both unicellular and multicellular organisms, with similar allosteric control of CDKs existing amongst multicellular organisms.

The present invention derives from the discovery and isolation of genes encoding novel cell-cycle regulatory proteins from the human fungal pathogen Candida. One benefit provided by the present invention derives from the use of the subject proteins, antibodies and nucleic acids as reagents for diagnostic assays. Conventional diagnosis, as indicated above, often involves time-consuming steps for determining the presence of infection. Such delays can be unacceptable where prompt treatment must be accorded in order to provide positive prognosis. The subject diagnostic assays, particularly PCR-based procedures, can provide diagnostically relevant information in shorter time periods.

Furthermore, in light of the expected indispensable role of each of these proteins in control of cell proliferation, the present invention specifically contemplates drug screening assays which detect agents that disrupt the activity of one or more of the subject regulatory proteins, such as by disruption of binding to other cellular proteins or, where applicable, by inhibition of an enzymatic activity of the protein. Agents which inhibit the activation of Candida CDKs can be used as anti-fungal agents, such as to treat mycotic infections in animals, as preservatives in foodstuff, as a feed supplement for promoting weight gain in livestock, or in disinfectant formulations for decontaminating equipment and rooms.

In particular, we have isolated from Candida genes which encode an apparent CDC25 phosphatase (“TYP1”), a p13^{suc1} homolog (“CKS1”), a cyclin dependent kinase (“CDK1”), a cyclin (“CYB1”), a CDK-activating kinase catalytic subunit (“MOC1”), and a Map kinase (“CMK1”). Each of these genes, while sharing some degree of homology with genes of other eukaryotes, are typically less than about 75 percent homologous with known genes, and many are less than 50 percent homologous with known genes. For convenience, Table 1 provides a guide to the relevant Sequence Listing entries which set forth the nucleic acid and amino acid sequences for the each of the subject regulatory genes.

Table 1: Sequence Listing Guide

| | nucleic acid | amino acid |
|---|---|---|
| clone | sequence | sequence |
| TYP1 | SEQ ID No. 1 | SEQ ID No. 7 |
| CKS1 | SEQ ID No. 2 | SEQ ID No. 8 |
| CDK1 | SEQ ID No. 3 | SEQ ID No. 9 |
| CYB1 | SEQ ID No. 4 | SEQ ID No. 10 |
| MOC1 | SEQ ID No. 5 | SEQ ID No. 11 |
| CMK1 | SEQ ID No. 6 | SEQ ID No. 12 |
| CAK1 | SEQ ID No. 13 | SEQ ID No. 14 |

For convenience, certain terms employed in the specification, examples, and appended claims are collected here.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

As used herein, the term “gene” or “recombinant gene” refers to a nucleic acid comprising an open reading frame encoding a polypeptide of the present invention, including both exon and (optionally) intron sequences. A “recombinant gene” refers to nucleic acid encoding such regulatory polypeptides, which may optionally include intron sequences which are either derived from a chromosomal DNA. Exemplary recombinant genes encoding the subject regulatory proteins are represented in SEQ ID Nos: 1-6 or 13. The term “intron” refers to a DNA sequence present in a given gene which is not translated into protein and is generally found between exons.

As used herein, the term “transfection” means the introduction of a nucleic acid, e.g., an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of a polypeptide of the present invention or where anti-sense expression occurs from the transferred gene, the expression of a naturally-occurring form of the protein is disrupted.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of the protein.

“Homology” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A “chimeric protein” or “fusion protein” is a fusion of a first amino acid sequence encoding one of the subject polypeptides with a second amino acid sequence defining a domain foreign to and not substantially homologous with any domain of the first polypeptide. A chimeric protein may present a foreign domain which is found (albeit in a different protein) in an organism which also expresses the first protein, or it may be an “interspecies”, “intergenic”, etc. fusion of protein structures expressed by different kinds of organisms.

The term “evolutionarily related to”, with respect to nucleic acid sequences encoding each of the regulatory proteins, refers to nucleic acid sequences which have arisen naturally in an organism, including naturally occurring mutants. The term also refers to nucleic acid sequences which, while derived from naturally occurring genes, have been altered by mutagenesis, as for example, combinatorial mutagenesis described below, yet still encode polypeptides which have at least one activity of the wild-type (“authentic”) protein.

The term “isolated” as also used herein with respect to nucleic acids, such as DNA or RNA, refers to molecules separated from other DNAs, or RNAs, respectively, that are present in the natural source of the macromolecule. For example, isolated nucleic acids encoding the subject polypeptides preferably include no more than 10 kilobases (kb) of nucleic acid sequence which naturally immediately flanks that gene in genomic DNA, more preferably no more than 5 kb of such naturally occurring flanking sequences, and most preferably less than 1.5 kb of such naturally occurring flanking sequence. The term isolated as used herein also refers to a nucleic acid or polypeptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state.

As described below, one aspect of this invention pertains to an isolated nucleic acid comprising the nucleotide sequence encoding one of the subject regulatory proteins, biologically active fragments thereof, and/or equivalents of such nucleic acids. The term nucleic acid as used herein is intended to include such fragments and equivalents. Moreover, the term “nucleic acid encoding a TYP1 phosphate” is understood to include nucleotide sequences encoding homologous proteins functionally equivalent to the polypeptides Candida, TYP1 protein set forth in SEQ ID No. 7, or functionally equivalent polypeptides which, for example, retain a phosphatase activity, and which may additionally retain other activities of a TYP1 protein, e.g., the ability to bind to a CDK, e.g. a CDK1.

In similar fashion, the present invention contemplates nucleic acids which encode polypeptides that are homologous and functionally equivalent to other of the subject regulatory proteins. For instance, an equivalent polypeptide of CKS1 may retain the ability to bind to CDK1.

An equivalent polypeptide of CDK1 can retain the ability to bind to cyclins, such as CYB1, as well as MOC1 and the like, TYP1, CKS1 and/or other regulatory proteins, as well as cellular substrates of the authentic form of the kinase. In addition, an equivalent CDK1 polypeptide may retain its kinase activity. In similar fashion, an equivalent MOC1 polypeptide may be characterized by binding to CDK1 or another cyclin-dependent kinase, as well as, or alternatively, by its kinase activity towards substrates of the naturally occurring form of the protein. Equivalent polypeptides of the subject CYB1 protein will typically retain the ability to bind to a CDK, e.g. CDK1.

Moreover, it will be understood that such equivalent polypeptides as described above may mimic (agonize) the actions of the authentic form of one of the subject regulatory proteins. However, it is expressly provided that such equivalents include polypeptides which function to antagonize the normal function of the wild-type protein. For instance, dominant negative mutants of any of the enzymes TYP1, CDK1, MOC1 or CMK1 may competitively inhibit the function of the authentic protein by binding to substrate without catalytically acting upon it. Mutants of any of the subject proteins which produce non-productive complexes with other regulatory proteins can likewise be antagonistic homologs. Accordingly, the term “biological activity”, with respect to homologs of the proteins enumerated in the Sequence Listing, refers to both agonism and antagonism of the ordinary function of the wild-type form of that protein.

Thus, equivalent nucleotide sequences will include sequences that differ by one or more nucleotide substitutions, additions or deletions, such as intragenus variants; and will also include sequences that differ from the nucleotide sequence encoding the portion of the a protein represented in one of SEQ ID Nos. 1-6 or 13 due to the degeneracy of the genetic code. Equivalent nucleic acids will also include nucleotide sequences that hybridize under stringent conditions (i.e., equivalent to about 20-27° C. below the melting temperature (Tₘ) of the DNA duplex formed in about 1 M salt) to a nucleotide sequence of a Candida gene represented in one of SEQ ID Nos. Nos. 1-6 or 13.

Preferred nucleic acids encode polypeptides comprising an amino acid sequence which is at least 60% homologous, more preferably 70% homologous and most preferably 80% homologous with an amino acid sequence shown in one of SEQ ID Nos. 7-12 or 14. Nucleic acids encoding polypeptides, particularly polypeptides retaining an activity of one of the subject regulatory proteins, and comprising an amino acid sequence which is at least about 90%, more preferably at least about 95%, and most preferably at least about 98-99% homologous with a sequence shown in one of SEQ ID Nos. 7-12 or 14 are also within the scope of the invention.

In yet a further embodiment, the recombinant regulatory genes may further include, in addition to the nucleic acid sequences shown in SEQ ID Nos. 1-6 or 13, additional nucleotide sequences. For instance, the recombinant gene can include nucleotide sequences of a PCR fragment generated by amplifying the gene from a genomic DNA library, e.g., intronic sequences, as well as 5′ and 3′ non-coding sequences of any of the subject genes.

Another aspect of the invention provides nucleic acid that hybridizes under high or low stringency conditions to nucleic acid which encodes a polypeptide identical or homologous with an amino acid sequence represented in one of SEQ ID Nos. 7-12 or 14. Appropriate stringency conditions which promote DNA hybridization, for example, 6.0×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6 or 13.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C.

Isolated nucleic acids encoding a Candida regulatory protein of the present invention, yet which differ from the nucleotide sequences shown in SEQ ID Nos. 1-6 or 13 due to degeneracy in the genetic code, are also within the scope of the invention. Such nucleic acids are understood to be capable of encoding functionally equivalent polypeptides (i.e., a polypeptide having at least a portion of the biological activity of a protein encoded by the enumerated sequences). For instance, a number of amino acids are designated by more than one triplet. Codons that specify the same amino acid (for example, CAU and CAC are synonyms for histidine) may result in “silent” mutations which do not affect the amino acid sequence of the protein. However, it is expected that DNA sequence polymorphisms that do lead to changes in the amino acid sequences of the protein will exist even within the same species. One skilled in the art will appreciate that these variations in one or more nucleotides (up to about 3-4% of the nucleotides) of a gene encoding a protein may exist among individual cells of a given species, e.g., amongst a population of C. albicans cells, due to natural allelic variation. Any and all such nucleotide variations and resulting amino acid polymorphisms are within the scope of this invention.

Fragments of the nucleic acid encoding portions of the subject regulatory proteins, such as the catalytic domain of the TYP1 phosphatase, are also within the scope of the invention. As used herein, such fragments refer to nucleotide sequences having fewer nucleotides than the coding sequence of the gene, yet still include enough of the coding sequence so as to encode a polypeptide with at least some of the activity of the full-length protein activity.

Nucleic acids within the scope of the invention may also contain linker sequences, modified restriction endonuclease sites and other sequences useful for molecular cloning, expression or purification of the recombinant polypeptides.

As indicated by the examples set out below, a nucleic acid encoding one of the subject proteins may be obtained from mRNA present in the cells of a pathogen from the genus Candida. It will also be possible to obtain nucleic acids encoding the subject proteins from genomic DNA obtained from such cells. For example, a gene encoding one of the pathogen regulatory proteins can be cloned from either a cDNA or a genomic library from other Candida species in accordance with protocols described herein, as well as those generally known in the art. For instance, a cDNA encoding a TYP1 protein can be obtained by isolating total mRNA from a culture of Candida cells, generating double stranded cDNAs from the total mRNA, cloning the cDNA into a suitable plasmid or bacteriophage vector, and isolating clones expressing TYP1 protein using any one of a number of known techniques, e.g., oligonucleotide probes or western blot analysis. Genes encoding related proteins can also be cloned using established polymerase chain reaction techniques in accordance with the nucleotide sequence information provided by the invention. The nucleic acid of the invention can be DNA or RNA.

Another aspect of the invention relates to the use of the isolated nucleic acid in “antisense” therapy. As used herein, “antisense” therapy refers to administration or in situ generation of oligonucleotide probes or their derivatives which specifically hybridizes (e.g. binds) under cellular conditions, with the cellular mRNA and/or genomic DNA encoding one of the subject regulatory proteins so as to inhibit expression of that protein, e.g. by inhibiting transcription and/or translation. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA which encodes one of the regulatory proteins. Alternatively, the antisense construct is an oligonucleotide probe which is generated ex vivo and which, when introduced into the cell, causes inhibition of expression by hybridizing with the complementary mRNA and/or genomic sequences. In any event, it will be generally desirable to choose an antisense molecule which uniquely hybridizes to the Candida gene, e.g. does not hybridize under physiological conditions to DNA or RNA from a mammalian cell, especially a human cell. Such oligonucleotide probes are preferably modified oligonucleotide which are resistant to endogenous nucleases, e.g. exonucleases and/or endonucleases, and is therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by van der Krol et al. (1988) Biotechniques 6:958-976; and Stein et al. (1988) Cancer Res 48:2659-2668.

Accordingly, the modified oligomers of the invention are useful in therapeutic, diagnostic, and research contexts. In therapeutic applications, the oligomers are utilized in a manner appropriate for antisense therapy in general. For such therapy, the oligomers of the invention can be formulated for a variety of modes of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous for injection, the oligomers of the invention can be formulated in liquid solutions, preferably in physiologically compatible buffers such as Hank's solution or Ringer's solution. In addition, the oligomers may be formulated in solid form and redissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the oligomers are formulated into conventional oral administration forms such as capsules, tablets, and tonics. For topical administration, the oligomers of the invention are formulated into ointments, salves, gels, or creams as generally known in the art.

In addition to use in therapy, the oligomers of the invention may be used as diagnostic reagents to detect the presence or absence of the target DNA or RNA sequences to which they specifically bind, e.g. to provide a diagnostic screen for fungicemia. In particular, because of the significant difference in sequence between the subject Candida nucleic acids and apparent orthologs of other eukaryotes, even other single cell eukaryotes, the probe/primer of the present invention will permit diagnostic assays which can rapidly distinguish Candida infection from other causative agents of fungicemia.

This invention also provides expression vectors which include a nucleotide sequence encoding one of the subject polypeptides and operably linked to at least one regulatory sequence. Operably linked is intended to mean that the nucleotide sequence is linked to a regulatory sequence in a manner which allows expression of the nucleotide sequence. Regulatory sequences are art-recognized. Accordingly, the term regulatory sequence includes promoters, enhancers and other expression control elements. Exemplary regulatory sequences are described in Goeddel; Gene Expression Technology: Methods in Enzymology 185, Academic Press, San Diego, Calif. (1990). For instance, any of a wide variety of expression control sequences-sequences that control the expression of a DNA sequence when operatively linked to it may be used in these vectors to express DNA sequences encoding the regulatory proteins of this invention. Such useful expression control sequences, include, for example, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other proteins encoded by the vector, such as antibiotic markers, should also be considered.

This invention also pertains to a host cell transfected with a recombinant gene in order that it may express a recombinant protein of the present invention. The host cell may be any prokaryotic or eukaryotic cell. For example, a TYP1 protein of the present invention may be expressed in bacterial cells, such as E. coli, insect cells, yeast, or mammalian cells. Other suitable host cells are known to those skilled in the art. Exemplary cells genetically engineered to produce a recombinant protein of the present invention are the Schizosaccharomyces cells described below.

Another aspect of the present invention concerns recombinant forms of the subject Candida regulatory proteins. The term “recombinant protein” refers to a protein of the present invention which is produced by recombinant DNA techniques, wherein generally DNA encoding the protein is inserted into a suitable expression vector which is in turn used to transform a host cell to produce the heterologous protein. Moreover, the phrase “derived from”, with respect to a recombinant gene encoding one of the subject proteins, is meant to include within the meaning of “recombinant protein” those proteins having an amino acid sequence of the native (or “authentic”) form of the pathogen protein, or an amino acid sequence similar thereto, which is generated by mutation so as to include substitutions and/or deletions relative to a naturally occurring form of the protein. To illustrate, recombinant proteins preferred by the present invention, in addition to those having an amino acid sequence of the native proteins, are those recombinant proteins having amino acid sequences which are at least 70% homologous, more preferably 80% homologous and most preferably 90% homologous with an amino acid sequence shown in one of SEQ ID Nos: 7-12 or 14. A polypeptide which having an amino acid sequence that is at least about 95%, more preferably at least about 98%, and most preferably identical to one of the sequences shown in SEQ ID Nos: 7-12 or 14 are also within the scope of the invention. Thus, the present invention pertains to recombinant proteins which are derived from Candida and which have amino acid sequences evolutionarily related to a protein represented by any one of SEQ ID Nos: 7-12 or 14, wherein “evolutionarily related to” refers to polypeptides having amino acid sequences which have arisen naturally (e.g. by allelic variance or by differential splicing), as well as mutational variants of the regulatory proteins which are derived, for example, by combinatorial mutagenesis.

The present invention further pertains to methods of producing the subject polypeptides. For example, a host cell transfected with a nucleic acid vector directing expression of a nucleotide sequence encoding one of the subject regulatory proteins can be cultured under appropriate conditions to allow expression of the polypeptide to occur. The polypeptide may be secreted and isolated from a mixture of cells and medium containing the recombinant protein. Alternatively, the polypeptide may be retained cytoplasmically and the cells harvested, lysed and the protein isolated. A cell culture includes host cells, media and other byproducts. Suitable media for cell culture are well known in the art. The polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for the recombinant protein. In a preferred embodiment, the regulatory protein is a fusion protein containing a domain which facilitates its purification, such as a GST fusion protein.

Thus, a nucleotide sequence derived from the cloning of one of the subject proteins, encoding all or a selected portion of the protein, can be used to produce a recombinant form of the protein via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial cells), are standard procedures used in producing other well-known intracellular proteins, e.g., p53, RB, p16, human TYP1, and the like. Similar procedures, or modifications thereof, can be employed to prepare recombinant forms of the subject proteins, or portions thereof, by microbial means or tissue-culture technology in accord with the subject invention.

Recombinant forms of the subject regulatory proteins can be produced by ligating the cloned gene, or a portion thereof, into a vector suitable for expression in either prokaryotic cells, eukaryotic cells, or both. Expression vehicles for production of the recombinant proteins include plasmids and other vectors. For instance, suitable vectors for the expression of the recombinant protein include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, pRS vectors, e.g., pRS303, pRS304, pRS305, pRS306, etc., are cloning and expression vehicles useful in the introduction of genetic constructs into S. cerevisiae (see, for example, Sikorski et al. (1989) Genetics 122:19-27; and Christianson (1992) Gene 110:119-122). These vectors can replicate in E. coli due to the presence of the pBR322 ori, and in S. cerevisiae due to the replication determinant of the yeast 2 micron plasmid. In addition, drug resistance markers such as ampicillin can be used. Expression in other yeast systems, such as P. pastoris, is contemplated by this invention.

In some instances, it may be desirable to express the recombinant genes by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the 13-gal containing pBlueBac III, p2Blue). Further, the p2Blue vector has the added feature of being capable of expressing two exogenous proteins simultaneously (p2Blue, Invitrogen Corp. Catalog number V-1970-10).

When expression of a carboxy-terminal portion of one of the polypeptides enzyme is desired, i.e., a truncated form of the protein, it may be desirable to add a start codon (ATG) to the oligonucleotide fragment containing the desired sequence to be expressed. It is well known in the art that a methionine at the N-terminal position can be enzymatically cleaved by the use of the enzyme methionine aminopeptidase (MAP). MAP has been cloned from E. coli (Ben-Bassat et al. (1987) J. Bacteriol. 169:751-757) and Salmonella typhimurium and its in vitro activity has been demonstrated on recombinant proteins (Miller et al. (1987) PNAS 84:2718-1722). Therefore, removal of an N-terminal methionine, if desired, can be achieved either in vivo by expressing recombinantly-derived polypeptides in a host which produces MAP (e.g., E. coli or CM89 or S. cerevisiae), or in vitro by use of purified MAP (e.g., procedure of Miller et al.).

Alternatively, the coding sequences for the polypeptide can be incorporated as a part of a fusion gene so as to be covalently linked in-frame with a second nucleotide sequence encoding a different polypeptide. This type of expression system can be useful, for instance, where it is desirable to produce an immunogenic fragment of the protein. For example, the VP6 capsid protein of rotavirus can be used as an immunologic carrier protein for portions of the TYP1 polypeptide, either in the monomeric form or in the form of a viral particle. The nucleic acid sequences corresponding to the portion of the protein to which antibodies are to be raised can be incorporated into a fusion gene construct which includes coding sequences for a late vaccinia virus structural protein to produce a set of recombinant viruses expressing fusion proteins comprising a portion of the TYP1 protein as part of the virion. It has been demonstrated with the use of immunogenic fusion proteins utilizing the Hepatitis B surface antigen fusion proteins that recombinant Hepatitis B virions can be utilized in this role as well. Similarly, chimeric constructs coding for fusion proteins containing a portion of a TYP1 protein and the poliovirus capsid protein can be created to enhance immunogenicity of the set of polypeptide antigens (see, for example, EP Publication No. 0259149; and Evans et al. (1989) Nature 339:385; Huang et al. (1988) J. Virol. 62:3855; and Schlienger et al. (1992) J. Virol. 66:2).

The Multiple Antigen Peptide system for peptide-based immunization can also be utilized, wherein a desired portion of a one of the subject proteins is obtained directly from organo-chemical synthesis of the peptide onto an oligomeric branching lysine core (see, for example, Posnett et al. (1988) JBC 263:1719 and Nardelli et al. (1992) J. Immunol. 148:914). Antigenic determinants of the subject proteins can also be expressed and presented by bacterial cells.

In addition to utilizing fusion proteins to enhance immunogenicity, it is widely appreciated that fusion proteins can also facilitate the expression of proteins. For example, recombinant forms of each of the subject pathogen proteins can be generated as glutathione-S-transferase (GST) fusion proteins. Such GST fusion proteins can be used to simplify purification of the protein, such as through the use of glutathione-derivatized matrices (see, for example, Current Protocols in Molecular Biology, eds. Ausabel et al. (N.Y.: John Wiley & Sons, 1991)). In another embodiment, a fusion gene coding for a purification leader sequence, such as a poly-(His)/enterokinase cleavage site sequence at the N-terminus of the desired portion of the recombinant protein, can facilitate purification of the fusion protein by affinity chromatography using a Ni²+metal resin. The purification leader sequence can then be subsequently removed by treatment with enterokinase (e.g., see Hochuli et al. (1987) J. Chromatography 411:177; and Janknecht et al. PNAS 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausabel et al. John Wiley & Sons: 1992).

The present invention also makes available purified, or otherwise isolated forms of the subject fungal proteins, which are isolated from, or otherwise substantially free of, other intracellular proteins which may be normally associated, especially other cell-cycle regulatory proteins. The term “substantially free of other cellular proteins” (also referred to herein as “contaminating proteins”) is defined as encompassing, for example, protein preparations comprising less than 20% (by dry weight) contaminating protein, and preferably comprises less than 5% contaminating protein. Purified forms of the subject polypeptides can be prepared as purified preparations, for example, by using the cloned genes as described herein. The term “purified” as used herein preferably means at least 80% by dry weight, more preferably in the range of 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above. “Isolated” and “purified” do not encompass either natural materials in their native state or natural materials that have been separated into components (e.g., in an acrylamide gel) but not obtained either as pure (e.g. lacking contaminating proteins, or chromatography reagents such as denaturing agents and polymers, e.g. acrylamide or agarose) substances or solutions.

However, the subject polypeptides can also be provided in pharmaceutically acceptable carriers for formulated for a variety of modes of administration, including systemic and topical or localized administration. Techniques and formulations generally may be found in Remmington's Pharmaceutical Sciences, Meade Publishing Co., Easton, Pa. In an exemplary embodiment, a dominant negative mutant of one of the subject regulatory proteins can be provided for transmucosal or transdermal delivery. For such administration, penetrants appropriate to the barrier to be permeated are used in the formulation with the polypeptide. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the oligomers of the invention are formulated into ointments, salves, gels, or creams as generally known in the art.

Another aspect of the invention related to polypeptides derived from the full-length forms of the subject proteins. Isolated peptidyl portions can be obtained by screening polypeptides recombinantly produced from the corresponding fragment of the nucleic acid encoding such polypeptides. In addition, fragments can be chemically synthesized using techniques known in the art such as conventional Merrifield solid phase f-Moc or t-Boc chemistry. For example, TYP1 can be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of, for example, CDK activation, such as by microinjection assays. In an illustrative embodiment, peptidyl portions of the Candida TYP1 can be tested for CDK-binding activity, as well as inhibitory ability, by expression as, for example, thioredoxin fusion proteins, each of which contains a discrete fragment of the TYP1 protein (see, for example, U.S. Pat. Nos. 5,270,181 and 5,292,646; and PCT publication WO94/02502).

It is also possible to modify the structure of the subject regulatory proteins for such purposes as enhancing therapeutic or prophylactic efficacy, or stability (e.g., ex vivo shelf life and resistance to proteolytic degradation in vivo). Such modified polypeptides, when designed to retain at least one activity of the naturally-occurring form of the protein, are considered functional equivalents of the polypeptides described in more detail herein. Such modified polypeptides can be produced, for instance, by amino acid substitution, deletion, or addition.

Moreover, it is reasonable to expect that an isolated replacement of a leucine with an isoleucine or valine, an aspartate with a glutamate, a threonine with a serine, or a similar replacement of an amino acid with a structurally related amino acid (i.e., conservative mutations) will not have a major effect on the biological activity of the resulting molecule. Conservative replacements are those that take place within a family of amino acids that are related in their side chains. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur containing=cysteine and methionine. (see, for example, Biochemistry, 2nd ed, Ed. by L. Stryer, W H Freeman and Co.:1981). Whether a change in the amino acid sequence of a peptide results in a functional homolog of one of the subject proteins can be readily determined by assessing the ability of the variant peptide to produce a response in cells in a fashion similar to the wild-type protein, or by evaluating the homolog in an in vitro system. Polypeptides in which more than one replacement has taken place can readily be tested in the same manner.

This invention further contemplates a method of generating sets of combinatorial mutants of the subject proteins, as well as truncation mutants, and is especially useful for identifying functional variant sequences. One purpose for generating and screening such combinatorial libraries is, for example, to isolate homologs from the library which function in the capacity as one of either an agonists or an antagonist of the biological activities of the authentic protein, or alternatively, which possess novel biological activities all together. To illustrate, TYP1 homologs can be engineered by the present method to provide homologs which lack phosphatase activity yet still retain the ability to bind to a CDK, e.g., a CDK1 binding capacity, or which bind to other cell-cycle proteins and prevent the action of the naturally occurring form of the protein. Such mutants can therefore be dominant negative phenotypes of the subject pathogen TYP1 enzyme, and can be used in, for example, gene therapy protocols that target delivery of a recombinant gene encoding a dominant negative TYP1 mutant to a pathogen.

For example, a combinatorial TYP1 library is produced by way of a degenerate library of genes encoding a library of polypeptides which each include at least a portion of potential TYP1 nucleotide sequences. A mixture of synthetic oligonucleotides can be enzymatically ligated into gene sequences such that the degenerate set of TYP1 nucleotide sequences are expressible as individual polypeptides, or alternatively, as a set of larger fusion proteins (e.g., for phage display) containing the TYP1 sequence library therein. In an illustrative embodiment, the library of TYP1 phosphatase mutants is expressed in the S. pombe cdc25-22, wee1-50 strain described below. Co-expression of the wild-type TYP1 (e.g. a recombinantly produced TYP1 from Candida, with a member of the TYP1 variant library, in conjunction with detecting proliferation of the cells, will permit the identification of dominant negative TYP1 mutants which are able to rescue the otherwise hyper-mitotic cell.

There are many ways by which the library of TYP1 homologs can be generated from a degenerate oligonucleotide sequence. For instance, chemical synthesis of a degenerate gene sequence can be carried out in an automated DNA synthesizer, and the synthetic genes then ligated into an appropriate gene for expression. The purpose of a degenerate set of TYP1 oligonucleotide sequences is to provide, in one mixture, all of the sequences encoding the desired set of potential TYP1 sequences. The synthesis of degenerate oligonucleotides is well known in the art (see, for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier polypeptide273-289; Itakura et al. (1984) Annu. Rev. Biochem. 53:323; Itakura et al. (1984) Science 198:1056; Ike et al. (1983) Nucleic Acid Res. 11:477). Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Moreover, there are several forms of mutagenesis generally applicable, in addition to a general combinatorial mutagenesis approach. For example, homologs of the subject proteins (both agonist and antagonist forms) can be generated and screened using, for example, alanine scanning mutagenesis and the like (Ruf et al. (1994) Biochemistry 33:1565-1572; Wang et al. (1994) J Biol Chem 269:3095-3099; Balint et al. (1993) Gene 137:109-118; Grodberg et al. (1993) Eur J Biochem 218:597-601; Nagashima et al. (1993) J Biol Chem 268:2888-2892; Lowman et al. (1991) Biochemistry 30:10832-10838; and Cunningham et al. (1989) Science 244:1081-1085), by linker scanning mutagenesis (Gustin et al. (1993) Virology 193:653-660; Brown et al. (1992) Mol Cell Biol 12:2644-2652; McKnight et al. (1982) Science 232:316); or by saturation mutagenesis (Meyers et al. (1986) Science 232:613). Such techniques will be generally understood to provides for reduction of the subject regulatory proteins to generate mimetics, e.g. peptide or non-peptide agents, which are able to disrupt binding of a naturally-occurring form of a protein of the present invention with other cell-cycle regulatory proteins of the pathogen from which it was derived, e.g. disrupts the binding of the pathogen TYP1 to a CDK.

Thus, such mutagenic techniques as described above are particularly useful to map the determinants of the subject proteins which participate in protein-protein interactions. To illustrate, the critical residues of a TYP1 protein which are involved in molecular recognition of a cyclin-dependent kinase, such as CDK1, can be determined and used to generate TYP1-derived peptidomimetics which competitively inhibit binding of the phosphatase with the CDK (see, for example, “Peptide inhibitors of human papillomavirus protein binding to retinoblastoma gene protein” European patent applications EP-412,762A and EP-B31,080A). By employing, for example, scanning mutagenesis to map the amino acid residues of one of the subject TYP1 involved in binding E6, peptidomimetic compounds (e.g. diazepine or isoquinoline derivatives) can be generated which mimic those residues in binding to CDK, and which therefore can inhibit binding of authentic TYP1 to CDK and thereby interfere with the function of TYP1 and/or the Kinase in proliferation of the pathogen. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted γ-lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), and β-aminoalcohols (Gordon et al. (1985) Biochem Biophys Res Commun 126:419; and Dann et al. (1986) Biochem Biophys Res Commun 134:71). In similar fashion, mimetics can be designed which bind to any of the other subject regulatory proteins, or mimic their binding to other proteins.

Another aspect of the invention pertains to antibodies and antibody preparations specifically reactive with at least one of the subject proteins. For example, by using peptides based on the cDNA sequence of one of the proteins represented in SEQ ID Nos. 7-12 or 14, anti-protein/anti-peptide antisera or monoclonal antibodies can be made using standard methods. A mammal such as a mouse, a hamster or rabbit, can be immunized with an immunogenic form of the peptide. Techniques for conferring immunogenicity on a protein or peptide include conjugation to carriers or other techniques well known in the art. An immunogenic form of the protein can be administered in the presence of adjuvant. The progress of immunization can be monitored by detection of antibody titers in plasma or serum. Standard ELISA or other immunoassays can be used with the immunogen as antigen to assess the levels of antibodies. In a preferred embodiment, the subject antibodies are immunospecific for antigenic determinants of one of the pathogen-derived proteins of the present invention, e.g. antigenic determinants of a protein represented by one of SEQ ID Nos. 7-12 or 14 or a closely related homolog (e.g. 90 percent homologous, more preferably at least 95 percent homologous). In yet a further preferred embodiment of the present invention, antibodies do not substantially cross react (i.e. do not react specifically) with a protein which is: e.g. less than 90 percent homologous, more preferably less than 95 percent homologous, and most preferably less than 98-99 percent homologous with one of SEQ ID Nos. 7-12 or 14. By “not substantially cross react”, it is meant that the antibody has a binding affinity for a nonhomologous protein, particularly orthologous proteins from mammalian cells, which is at least one order of magnitude, more preferably at least two orders of magnitude, and even more preferably at least three orders of magnitude less than the binding affinity of that antibody for one of the proteins of SEQ ID Nos. 7-12 or 14.

An effective amount of a conjugate-containing composition is introduced into a host animal such as a goat, rabbit, mouse, rat, horse or the like to induce the production (secretion) of antibodies to the polypeptide. Effective amounts of immunogens useful for inducing antibody secretions in host animals are well known in the art. Methods of introduction into the host animal are also well known and are typically carried out by parental administration as by injection. A plurality of such introductions is normally utilized so that the host is hyperimmunized to the immunogenic polypeptide-containing conjugate. For example, weekly introductions over a one-to-two-month time period can be utilized until a desired anti-polypeptide antibody titer is achieved.

Following immunization antisera can be obtained and, if desired, polyclonal antibodies isolated from the serum. To produce monoclonal antibodies, antibody producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. polypeptide. 77-96). Hybridoma cells can be screened immunochemically for production of antibodies specifically reactive with the immunogen and monoclonal antibodies isolated from a culture comprising such hybridoma cells.

The term antibody as used herein is intended to include fragments thereof which are also specifically reactive with one of the subject proteins. Antibodies can be fragmented using conventional techniques and the fragments screened for utility in the same manner as described above for whole antibodies. For example, F(ab′)₂ fragments can be generated by treating a full antibody with pepsin. The resulting F(ab′)₂ fragment can be treated to reduce disulfide bridges to produce Fab′ fragments. The antibody of the present invention is further intended to include bispecific and chimeric molecules.

An antibody preparation of this invention prepared from a polypeptide as described above can be in dry form as obtained by lyophilization. However, the antibodies are normally used and supplied in an aqueous liquid composition in serum or a suitable buffer such as PBS.

Both monoclonal and polyclonal antibodies (Ab) directed against one of the subject regulatory proteins, and antibody fragments such as Fab′ and F(ab′)₂, can be used to block the action of that protein and allow the study of its role in the cell-cycle or in cell proliferation. Moreover, such antibodies can also be used diagnostically to detect an infection involving Candida.

Moreover, the nucleotide sequence determined from the cloning of the subject regulatory proteins will permit the generation of probes designed for use in identifying the presence of a Candida infection such as an infection involving C. albicans. For instance, the present invention provides a probe/primer comprising a substantially purified oligonucleotide, wherein the oligonucleotide comprises a region of nucleotide sequence which hybridizes under stringent conditions to at least 10, more preferably 25, 50, or 100 consecutive nucleotides of sense or anti-sense sequence of one of SEQ ID Nos: 1-6 or 13, or naturally occurring mutants thereof. In preferred embodiments, the probe/primer further comprises a label group attached thereto and able to be detected, e.g. the label group is selected from the group consisting of radioisotopes, fluorescent compounds, enzymes, and enzyme co-factors. Such probes can be used as a part of a diagnostic test kit for identifying and phenotyping particular mycotic infections, such as in a sample of cells from a patient, or in a foodstuff, or on equipment.

The present invention also provides assays and reagents for identifying anti-fungal and anti-parasitic agents, e.g. agents which act to inhibit proliferation of a pathogen by altering the activity of one or more of the subject pathogen proteins. To illustrate, inhibitors of the Candida TYP1 phosphatase can be used in the treatment of candidiasis an opportunistic infection that commonly occurs in debilitated and immunosuppressed patients. TYP1 inhibitors could be used to treat these infections in patients with leukemias and lymphomas, in people who are receiving immunosuppressive therapy, and in patients with such predisposing factors as diabetes mellitus or AIDS, where fungal infections are a particular problem. TYP1 inhibitors can be generated for treatment of mycotic infections caused by, for example, Candida albicans, Candida stellatoidea, Candida tropicalis, Candida parapsilosis, Candida krusei, Candida pseudotropicalis, Candida quillermondii, Candida glabrata, Candida lusianiae, or Candida rugosa. Anti-proliferative agents developed with the subject assays can also be used, for example, as preservatives in foodstuff, as a feed supplement for promoting weight gain in livestock, or in disinfectant formulations for treatment of non-living matter, e.g., for decontaminating hospital equipment and rooms. Furthermore, as a result of the considerable divergence between TYP1 proteins, it is likely that differential screening assays, e.g. side-by-side comparison of inhibition of human TYP1 relative to one of the Candida TYP1 enzyme, can be used to identify agents that exhibit specific inhibitory effects directed at the form of the subject TYP1 protein present in the pathogen, without substantially inhibiting a CDC25 phosphatase in human or other animal cells. Thus, by making available purified and recombinant proteins, the present invention facilitates the development of assays which can be used to screen for drugs which are either agonists or antagonists of the normal cellular function of the subject regulatory proteins. An inhibitor, as identified in the subject assays, is an agent which is able to cause a statistically significant decrease in one or more proliferative activities of a regulatory protein of the present invention.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target, as may be manifest in an alteration of binding affinity between one of the subject proteins and other proteins with which they interact, in changes in enzymatic activity of one of the subject proteins, or in changes in a property of the molecular target manifest from binding to one of the regulatory proteins.

Accordingly, in an exemplary screening assay of the present invention, the compound of interest is contacted with an isolated and purified TYP1 polypeptide which is ordinarily capable of binding a cyclin-dependent kinase. To the mixture of the compound and TYP1 polypeptide is then added a composition containing a CDK polypeptide. Detection and quantification of CDK/TYP1 complexes provides a means for determining the compound's efficacy at inhibiting (or potentiating) complex formation between the CDK and TYP1 polypeptides. The efficacy of the compound can be assessed by generating dose response curves from data obtained using various concentrations of the test compound. Moreover, a control assay can also be performed to provide a baseline for comparison. In the control assay, an isolated and purified CDK is added to a composition containing the TYP1 protein, and the formation of CDK/TYP1 complexes is quantitated in the absence of the test compound. Efficacy of an agent is based on producing a statistically significant change in formation of such complexes relative to the control. It will be understood that, in general, the order in which the reactants may be admixed can be varied, and can be admixed simultaneously.

Complex formation between the TYP1 polypeptide and CDK polypeptide may be detected by a variety of techniques. For instance, modulation of the formation of complexes can be quantitated using, for example, detectably labeled proteins such as radiolabelled (e.g. ³²P, ³⁵S, ¹⁴C or ³H), fluorescently labeled (e.g. FITC), or enzymatically labeled polypeptides, by immunoassay, or by chromatographic detection. The use of enzymatically labeled TYP1 or CDK proteins will, of course, generally be used only when enzymatically inactive portions of those proteins are used, as each protein can possess a measurable intrinsic activity which can be detected.

Typically, it will be desirable to immobilize one of the two polypeptides to facilitate separation of complexed from uncomplexed forms of one or both of the proteins, as well as to accommodate automation of the assay. Binding of the CDK to TYP1, in the presence and absence of a candidate agent, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/TYP1 (GST/TYP1) fusion proteins can be adsorbed onto glutathione sepharose beads (Sigma Chemical, St. Louis, Mo.) or glutathione derivatized microtitre plates, which are then combined with the CDK polypeptide, e.g. an ³⁵S-labeled CDK polypeptide, and the test compound, and the mixture incubated under conditions conducive to complex formation, e.g. at physiological conditions for salt and pH, though slightly more stringent conditions may be desired, e.g., at 4° C. in a buffer containing 0.6M NaCl or a detergent such as 0.1% Triton X-100. Following incubation, the beads are washed to remove any unbound CDK polypeptide, and the matrix immobilized radiolabel determined directly (e.g. beads placed in scintillant), or in the supernatant after the protein complexes are subsequently dissociated. Alternatively, the complexes can dissociated from the matrix, separated by SDS-PAGE, and the level of labeled polypeptide found in the bead fraction quantitated from the gel using standard electrophoretic techniques such as described in the appended examples.

Other techniques for immobilizing proteins on matrices are also available for use in the subject assay. For instance, either of the proteins can be immobilized utilizing conjugation of biotin and streptavidin. For instance, biotinylated TYP1 molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with the TYP1 but which do not interfere with CDK binding can be derivatized to the wells of the plate, and the TYP1 polypeptide trapped in the wells by antibody conjugation. As above, preparations of a CDK polypeptide and a test compound are incubated in the TYP1 presenting wells of the plate, and the amount of protein complex trapped in the well can be quantitated. Exemplary methods for detecting such complexes, in addition to those described above for the GST-immobilized complexes, include immunodetection of complexes using antibodies reactive with the CDK polypeptide, or which are reactive with the TYP1 protein and compete for binding with the CDK polypeptide; as well as enzyme-linked assays which rely on detecting an enzymatic activity associated with the CDK polypeptide (instead of the intrinsic activity). In the instance of the latter, the enzyme can be chemically conjugated or provided as a fusion protein with a CDK polypeptide. To illustrate, a CDK1 polypeptide can be chemically cross-linked or genetically fused with horseradish peroxidase, and the amount of CDK1 trapped in the complex with TYP1 can be assessed with a chromogenic substrate of the exogenous enzyme, e.g. 3,3′-diamino-benzadine terahydrochloride or 4-chloro-1-napthol. Likewise, a fusion protein comprising the CDK and glutathione-S-transferase can be provided, and complex formation quantitated by detecting the GST activity using 1-chloro-2,4-dinitrobenzene (Habig et al (1974) J Biol Chem 249:7130).

As alluded to above, intrinsic enzymatic activities can be relied upon to detect the efficacy of an agent against TYP1. The detection of the TYP1 phosphatase activity is described in more detail below. However, the downstream targets of TYP1, such as a CDK, may also have an intrinsic activity which can be utilized to quantitate the interaction with TYP1. In an exemplary embodiment, an enzymatically active TYP1 is contacted with a phosphorylated CDK/cyclin complex, e.g. CDK1/CYB1, under conditions wherein, absent an inhibitor of the TYP1, that enzyme would dephosphorylate and activate the CDK/cyclin complex. Activation could be detected by conversion of a substrate for the kinase complex, such as phosphorylation of a histone H1 protein with ³²P-labeled phosphate.

For processes which rely on immunodetection for quantitating one of the proteins trapped in the complex, antibodies against the protein, such as either anti-CDK or anti-TYP1 antibodies, can be used. Alternatively, the protein to be detected in the complex can be “epitope tagged” in the form of a fusion protein which includes, in addition to the CDK polypeptide or TYP1 sequence, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharamacia, NJ).

Moreover, the subject polypeptides can be used to generate an interaction trap assay, as described in the examples below (see also, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696), for subsequently detecting agents which disrupt binding of TYP1 to a CDK or other cell-cycle regulatory protein, such as a cyclin.

The interaction trap assay relies on reconstituting in vivo a functional transcriptional activator protein from two separate fusion proteins, one of which comprises the DNA-binding domain of a transcriptional activator fused to a binding partner of TYP1, such as a CDK. The second fusion protein comprises a transcriptional activation domain (e.g. able to initiate RNA polymerase transcription) fused to the TYP1 polypeptide. When the CDK and TYP1 domains of each fusion protein interact, the two domains of the transcriptional activator protein are brought into sufficient proximity as to cause transcription of a reporter gene. By detecting the level of transcription of the reporter, the ability of a test agent to inhibit (or potentiate) the interaction can be evaluated. Commercial kits for generating interaction traps are presently available (e.g., MATCHMAKER Kit, Clontech catalog No. k1605-1, Palo Alto) and, in light of the present disclosure, can be modified for use as drug screening assays.

In an illustrative embodiment, Saccharomyces cerevisiae YPB2 cells are transformed simultaneously with a plasmid encoding a GAL4 db-CDK fusion and with a plasmid encoding the GAL4ad domain fused to a the Candida TYP1. Moreover, the strain is transformed such that the GAL4-responsive promoter drives expression of a phenotypic marker. For example, the ability to grow in the absence of histidine can depends on the expression of the HIS3 gene. When the HIS3 gene is placed under the control of a GAL4-responsive promoter, relief of this auxotrophic phenotype indicates that a functional GAL4 activator has been reconstituted through the interaction of the CDK and the TYP1 proteins. Thus, a test agent able to inhibit this interaction will result in yeast cells unable to growth in the absence of histidine. Alternatively, the phenotypic marker (e.g. instead of the HIS3 gene) can be one which provides a negative selection (e.g., are cytotoxic) when expressed such that agents which disrupt CDK/TYP1 interactions confer positive growth selection to the cells.

It will be apparent that, in similar fashion to the exemplary TYP1-derived assays, each of the other Candida regulatory proteins can be used to generate equivalent drug screening assays which provide a protein-protein interaction as the inhibitory target. For example, each of the CYB1, MOC1 and CKS1 proteins can be used to generate assays for detecting agents which inhibit interaction with a CDK, such as CDK1.

Moreover, for each of the subject regulatory proteins which have intrinsic enzymatic activities, such as the TYP1, CDK1, MOC1 and CMK1 proteins, the present invention provides methods and reagents for identifying agents which inhibit the enzymatic activity of the protein, e.g. agents which are mechanism based inhibitors of the enzyme, rather than merely disrupting the formation of a protein complex. Inhibitors of the enzymatic activity can be identified, for example, using assays generated for measuring the ability of an agent to inhibit catalytic conversion of a substrate by one of the subject enzymes. Again using TYP1 as an illustrative embodiment, a molecule or compound (e.g. a “test agent”) to be assessed for its ability to inhibit the phosphatase activity of the subject TYP1 enzyme is combined with the enzyme and a substrate of its phosphatase activity. The resulting combination is maintained under conditions appropriate for the TYP1 enzyme to act upon the substrate. The conversion of the substrate to product by the subject TYP1 enzyme is assessed, and the result compared to the rate or level of conversion of the substrate in the absence of the test agent. A statistically significant decrease in the activity of the TYP1 phosphatase in the presence of the test agent, manifest as a decrease in conversion of substrate to product, indicates that the test agent is an inhibitor of the pathogen TYP1.

In preferred embodiments, the substrate of the TYP1 tyrosine phosphatase is a synthetic substrate, e.g. a peptide or tyrosine analog, comprising a calorimetric or fluorescent label which is detectable when the substrate is catalytically acted upon by the TYP1. As used herein “calorimetric” refers to substrates detectable by change in absorption or fluorescent characteristics. For instance, preferred synthetic substrates include p-nitrophenylphosphate (pNPP), fluorosceindiphosphate (FDP), 3-O-methylfluoroscein phosphate (3-MFP). Other chromogenic substrates include 3-(p-hydroxyphenyl) propionic acid (HPPA), 2-Naphthyl phosphate, pyridoxal phosphate, adamantyl 1,2-doxetance phosphate, disodium 3-(4-methoxyspirol {1,2-dioxetane-3,2′-(5′-chloro) tricyclo [3.3.1.1] decan}-4-yl) phenyl phosphate, Thymolphtalein monophosphate, 3-indoxyl phosphate and the like. Yet other substrates include radiolabeled peptides, such as peptides containing ³²P-labeled phosphotyrosines, e.g. tyrosine phosphorylated forms of reduced carboxamindomethylated, maleyated lyzosyme (RCML) or CDC-derived peptides, wherein release of the radiolabel can be detected and correlated with TYP1 enzymatic activity.

In an illustrative embodiment, the method comprises the steps of: (a) combining a compound to be assessed, the subject Candida TYP1 (purified or semipurified), and a synthetic substrate of the pathogen TYP1 tyrosine phosphatase comprising a colorimetric label which is detectable when the substrate is acted upon by the TYP1 (e.g., p-nitrophenylphosphate); (b) maintaining the substrate/enzyme/test compound combination under conditions appropriate for the pathogen-derived TYP1 to act upon the substrate; and (c) determining, by colorimetric assay, the extent to which the TYP1 enzyme present in the combination acted upon the substrate, relative to a control, the control comprising the TYP1 and the substrate. If the subject TYP1 enzyme acts upon the substrate to a lesser extent than in the control, the compound is an inhibitor of the pathogen TYP1 tyrosine phosphatase activity.

In yet another embodiment of the present invention, inhibitors of the subject regulatory proteins which are involved in positive growth regulations are identified through their ability to rescue an otherwise hyper-mitotic cell from mitotic catastrophe, e.g. such as described in U.S. patent application Ser. No. 08/073,383. The term hyper-mitotic cell denotes a cell having an impaired cell-cycle regulatory protein which can cause premature progression of the cell though at least a portion of the cell-cycle and ultimately resulting in cell death. The hyper-mitotic cell of the subject assay can be generated, for example, by disrupting expression of a gene whose product acts antagonistically to one of the subject proteins, by overexpressing one of the subject proteins, or a combination thereof. In preferred embodiments, the impaired checkpoint of the hyper-mitotic cell would, in normal cells, otherwise act as a negative regulator of downstream mitotic events induced by one of the regulatory proteins of the present invention. Impairment of such a negative regulator consequently allows the cell to proceed aberrantly toward subsequent mitotic stages and ultimately inhibits faithful proliferation of the cell. In the presence of an agent able to inhibit the function of the targeted regulatory protein, progression of the hyper-mitotic cell through the cell-cycle can be slowed to enable the cell to appropriately undergo mitosis and proliferate with fidelity.

The present assay therefore provides a simple and rapid screening test which relies on scoring for positive proliferation as indicative of agents able to inhibit the function of, for example, one of the Candida regulatory proteins of the present invention, e.g., TYP1, CDK1, CYB1 or MOC1. One advantage of the subject assay is that while direct inhibition of growth can be caused by any toxic compound added to a proliferating cell culture, growth stimulation in the present assay will only be achieved upon specific inhibition of the targeted regulatory protein. Another advantage of the present assay is the amenity of the assay to high throughput analysis.

With regard to the hyper-mitotic cell of the present assay, impairment of the regulatory protein can be generated so as to be either continual or conditional. A conditional impairment permits the checkpoint to be normatively operational under some conditions such that the cell may proliferate and be maintained by cell culture techniques; and be rendered inoperative, or alternatively hyper-operative, under other conditions. In the instance of the illustrative wee1-50 mutant described below, the impaired checkpoint is effectively inoperative to an extent that the impairment allows aberrant mitosis to occur which concludes in mitotic catastrophe. A continual impairment, on the other hand, is one that is ever-present and which allows proliferation of the cell under conditions where there is no need to halt the cell at that checkpoint; but, in the instance of the hyper-mitotic cell, results in mitotic catastrophe under conditions where the cell-cycle must be halted, such as in the presence of DNA synthesis inhibitors or DNA damaging agents.

Regulatory pathways which feed into and modulate the activity of a CDK, such as CDK1, can be manipulated to generate the hyper-mitotic cell of the present assay. For example, as set out above, the inhibitory phosphorylation of cyclin-dependent kinases is mediated by at least two tyrosine kinases, initially identified in fission yeast and known as wee1 and mik1 (Russell et al. (1987) Cell 49:559; Lundgren et al. (1991) Cell 64:111; Featherstone et al. (1991) Nature 349:808; and Parker et al. (1991) EMBO 10:1255). These kinases act as mitotic inhibitors, overexpression of which causes cells to arrest in the G2 phase of the cell-cycle. For instance, overexpression of wee1 has been shown to cause intense phosphorylation of CDC2 (CDC28 in budding yeast) which results in cell-cycle arrest. Conversely, loss of function of wee1 causes advancement of mitosis and cells enter mitosis at approximately half the normal size, whereas loss of wee1 and mik1 function causes grossly premature initiation of mitosis, uncoupled from all checkpoints that normally restrain cell division. Thus, wee1 and mik1, or homologs thereof, each represent suitable regulatory proteins which could be impaired to generate the hyper-mitotic cell of the present assay.

Furthermore, it is apparent that enzymes which modulate the activity of the wee1 or mik1 kinases can also be pivotal in controlling the precise timing of mitosis. For example, the level of the nim1/cdr1 protein, a negative regulator of the wee1 protein kinase, can have a pronounced impact on the rate of mitotic initiation, and nim1 mutants have been shown to be defective in responding to nutritional deprivation (Russel et al. (1987) Cell 49:569; and Feilotter et al. (1991) Genetics 127:309). Over-expression of nim1 (such as the S. pombe op-nim1 mutant) can result in inhibition of the wee1 kinase and allow premature progression into mitosis. In like manner, mutation in the stf1 gene has also been shown to relieve regulation of mitotic progression in response to DNA synthesis inhibition.

Loss-of-function strains, such as the S. Pombe wee1-50, or mik1::ura (Rowley et al. (1992) Nature 356:353), are well known. In addition, each of the wee1, mik1, and nim1 genes have been cloned (see for example Coleman et al. (1993) Cell 72:919; and Feilotter et al. (1991) Genetics 127:309), such that disruption of wee1 and/or mik1 expression or over-expression of nim1 can be carried out to create the hyper-mitotic cell of the present assay. In a similar fashion, over-expression of wee1 and/or mik1 or disruption of nim1 expression can be utilized to generate a hypo-mitotic cell.

The hyper-mitotic cell of the present assay can be generated by manipulation of the cell in which one of the subject regulatory proteins expressed, as for example, by generating a wee1 mutation (a “wee” phenotype), or by exposure of the cell to 2-aminopurine or caffeine after a γ-radiation induced G2 arrest. It is also deemed to be within the scope of this invention that the hyper-mitotic cells of the present assay can be generated so as to comprise genetically engineered cells which express recombinant (e.g. heterologous) forms of the subject proteins. For instance, each of the subject recombinant TYP1, CDK1, MOC1 and CYB1 genes can be expressed in cells other than Candida, but in which the Candida gene is able to rescue lack-of-function mutations of the orthologous activity is the host cell. For example, the subject TYP1 gene can be used to replace the endogenous CDC25 gene of a hyper-mitotic Schizosaccharomyces cell, such as an S. pombe cell like the temperature-sensitive cdc25-22, wee1-50 mutant described below.

Moreover, in addition to complementation of CDC25-defective cells with the subject TYP1, the reagent cells of the subject assay can be further engineered to also express other exogenous cell-cycle proteins which interact with TYP1, e.g. Candida CDK. In an illustrative embodiment, a hyper-proliferative cell in which a Candida TYP1 is exogenously expressed can also be engineered to produce a Candida CDK (CDK1) and (optionally) a Candida cyclin (such as CYB1) and/or a CAK (e.g. MOC1). In this manner, the reagent cells of the present assay can be generated to more closely approximate the natural interactions which the pathogen phosphatase might experience.

In other embodiments, manipulation of cell-cycle regulatory pathways with certain drugs, termed here “hyper-mitotic agents”, can induce mitotic aberrations and result in generation of the hyper-mitotic cell of the present assay. For instance, caffeine, the protein kinase inhibitors 2-aminopurine and 6-dimethylaminopurine, and the protein phosphatase inhibitor okadaic acid can cause cells that are arrested in S phase by DNA synthesis inhibitors to inappropriately enter mitosis (Schlegel et al. (1986) Science 232:1264; Schlegel et al. (1987) PNAS 84:9025; and Schlegel et al. (1990) Cell Growth Differ. 1:171). Further, 2-aminopurine is believed to be able to override a number of cell-cycle checkpoints from G1, S phase, G2, or mitosis. (Andreassen et al. (1992) PNAS 89:2272; Andreassen et al. (1991) J. Cell Sci. 100:299, and Steinmann et al. (1991) PNAS 88:6843). For example, 2-aminopurine permits cells to overcome a G2/M block induced by γ-irradiation. Additionally, cells continuously exposed to 2-aminopurine alone are able to exit S phase without completion of replication, and exit mitosis without metaphase, anaphase, or telophase events. The effect of inhibitors of, for example, TYP1 function can therefore act to slow the progression of the cell through the cell-cycle and, at appropriate concentrations, offset the effects of the hyper-mitotic agent so as to permit cell growth rather than mitotic catastrophe.

Furthermore, to aid in the facilitation of mitotic catastrophe in the hyper-mitotic cell it may be desirable to expose the cell to an agent (i.e., a chemical or environmental stimulus) which ordinarily induces cell-cycle arrest. Inappropriate exit from the chemically- or environmentally-induced arrested state due to the impairment of the negative regulatory checkpoint can ultimately be lethal to the cell. Such arresting agents can include exposure to DNA damaging radiation or DNA damaging agents; inhibition of DNA synthesis and repair using DNA polymerase inhibitors such as hydroxyurea or aphidicolin; topoisomerase inhibitors such as 4′-dimethly-epipodophyllotoxin (VM-26); or agents which interfere with microtubule-assembly, such as Nocadazole and taxol. By way of example, the wee1-50 S. pombe cells described below can be dosed with γ-radiation in the presence of either caffeine, 2-aminopurine, or 6-dimethyl-aminopurine. Each of these compounds can suppress a G2 mitotic delay ordinarily caused by irradiation, and allow the cells to undergo mitosis before DNA repair has been completed. Inhibition of TYP1 activation of a CDK/cyclin complex may result in an offsetting effect which slows cell-cycle progression such that, at appropriate concentrations, the TYP1 inhibitor would rescue the hyper-mitotic cell. Additionally, in certain cells, nutritional status of the cell, as well as mating factors, can cause arrest of the normal cell during mitosis.

Agents to be tested for their ability to act as inhibitors can be produced by bacteria, yeast or other organisms (e.g. natural products), produced chemically (e.g. small molecules, such as peptidomimetics), or produced recombinantly. The assay can be carried out in any vessel suitable for the growth of the cell, such as microtitre plates or petri dishes. As potent inhibitors of the subject proteins would be expected to fully inhibit cell-cycle progression of even the hyper-mitotic cells, it will typically be desirable to perform the assay at various concentrations of the candidate agent. For example, serial dilutions of the candidate agents can be added to the hyper-mitotic cell such that at least one concentration tested the anti-mitotic agent inhibits the regulatory protein to an extent necessary to adequately slow the progression of the cell through the cell-cycle, but not to the extent necessary to completely inhibit entry of the cell into mitosis all together.

Quantification of proliferation of the hyper-mitotic cell in the presence and absence of a candidate agent can be measured using a number of techniques well known in the art, including simple measurement of population growth curves. For instance, where the assay involves proliferation in a liquid medium, turbidimetric techniques can be utilized (e.g., absorbence/transmittance of light of a given wavelength through the sample). For example, in the embodiment wherein the reagent cell is a yeast cell, measurement of absorbence of light at a wavelength between 540 and 600 nm can provide a conveniently fast measure of cell growth. Likewise, ability to form colonies in solid medium (e.g., agar) can be used to readily score for proliferation. Both of these techniques, especially with respect to yeast cells, are suitable for high through-put analysis necessary for rapid screening of large numbers of candidate agents. In addition, the use of solid media, such as agar-based plates, can further aid in establishing a serial dilution of the candidate agent. For example, the candidate agent can be spotted on a lawn of reagent cells plated on solid media. The diffusion of the candidate agent through the solid medium surrounding the site at which it was spotted will create a diffusional effect. For agents which inhibit the targeted regulatory protein, a halo of cell growth would be expected in an area which corresponds to concentrations of the agent which merely offset the effect of the impaired checkpoint, but which are not so great as to over-compensate for the impairment or too little so as to be unable to rescue the cell.

To further illustrate, other proliferative scoring techniques useful in the present assay include measuring the mitotic index for untreated and treated cells; uptake of detectable nucleotides, amino acids or dyes; as well as visual inspection of morphological details of the cell, such as chromatin structure or other features which would be distinguishable between cells advancing appropriately through mitosis and cells concluding in mitotic catastrophe or stuck at certain cell-cycle checkpoint.

### Exemplification

The invention now being generally described, it will be more readily understood by reference to the following examples which are included merely for purposes of illustration of certain aspects and embodiments of the present invention, and are not intended to limit the invention.

As described herein, we have isolated and characterized several genes from Candida which encode proteins that regulate progress of the Candida cell through mitosis and/or meiosis. As described in example 1, a phosphatase, TYP1, was cloned from C. albicans, and determined to be related to the CDC25 phosphatase gene family. To validate the identity of the isolated gene, we demonstrate that it is able to rescue a temperature sensitive allele cdc25-22 of fission yeast. To salient features of the Candida TYP1 gene are: although the TYP1 gene has less than 50% homology with yeast cdc25 genes, and less than 10% homology with the human cdc25 genes, the enzyme apparently performs the same function in regulation of cell cycle progression. Furthermore, despite earlier reports that certain preparations of the cdc25 phosphatase would not hydrolyze synthetic substrates in vitro (see Gautier et al. (1991) Cell 67:197-211, recombinant forms (including bacterially expressed) of the Candida TYP1 enzyme are able to hydrolyze such substrates.

### EXAMPLE 1

### Cloning of Candida TYP1

In order to isolate a gene encoding a Candida TYP1 phosphatase, the degenerate oligonucleotides ATGGATCCYTTRTANCCNCCRTSNARNANRTANAYNTCNGGRTA, ATGGATCCATIATIGAYTGYMGITWYCCITAYGA, and ATGGATCCATIATIGAYTGYMGITWYGAITAYGA were used to amplify C. albicans genomic DNA in λZAP (strain 3153A) by standard PCR protocols. The PCR reaction products were separated on a 2.5% low melting agarose gel that identified a sizable fragment (approximately 250 BP). The fragment was cloned into the pCRII vector (TA cloning system, Invitrogen) and the nucleotide sequence confirmed the identity of the insert as a likely TYP1 phosphatase. DNA probes were generated as ³²P-labeled nick translation products of the fragment, and used to further screen C. albicans cDNA libraries. Larger cDNA clones were isolated by this technique, and sequenced. The sequence of the open reading frame of the Candida TYP1 gene is given in SEQ. ID. No. 1, which also includes both 5′ and 3′ non-coding sequences.

To validate the identity of the isolated cDNA, the TYP1 clone was tested for its ability to rescue the temperature sensitive allele cdc25-22 of the fission yeast. Briefly, a 1.2 kbp EcoRI insert containing most of the open reading frame but lacking the amino part was cloned into the SmaI site of the pART1 vector, the resulting vector being designated pART-TYP1. As described in the literature, e.g. see WO 94/28914, the pART1 vector contains the constitutive S. pombe ADH promoter, the ars1 fragment for replication and the S. cerevisiae LEU2 gene as a marker which complements the leu1-32 mutant in S. pombe. Transformants growing on medium lacking leucine were streaked on plates and transferred at permissive temperature (37° C.). It was observed that only the cells expressing the C. albicans gene were able to form colonies. Microscopic observations of the cells revealed the rescue from the cell elongation typical for this mutant at restrictive temperature.

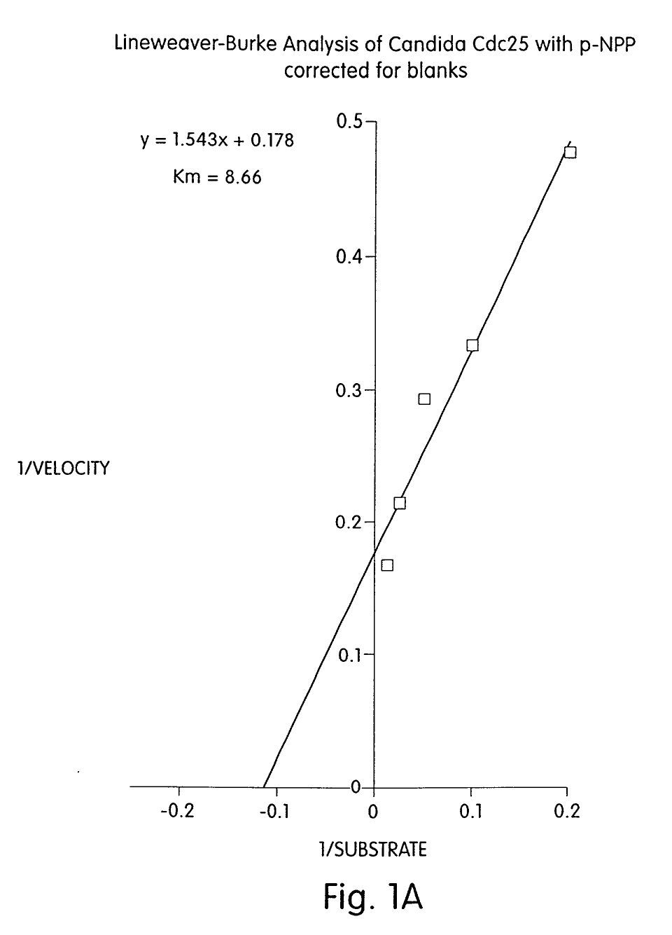

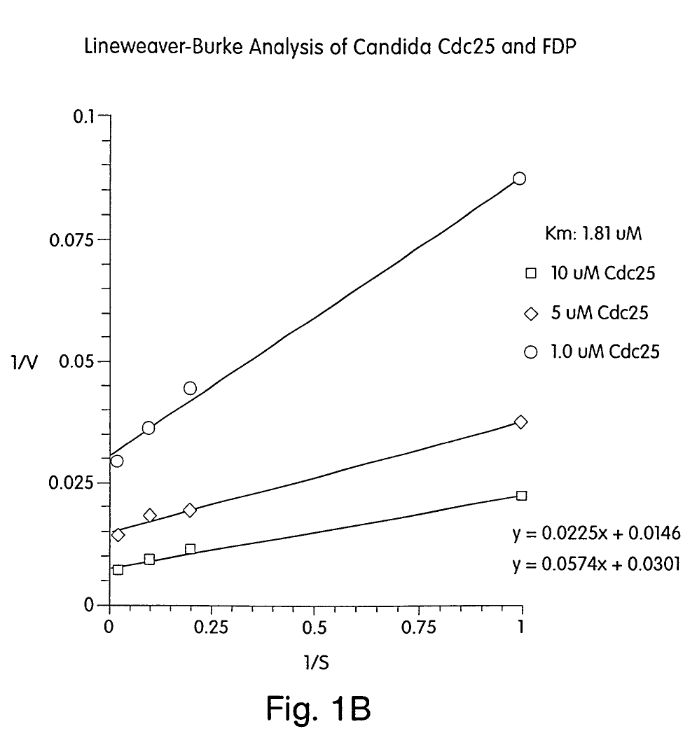

The C. albicans TYP1 cDNA gene was subsequently used to derive a fusion protein with glutathione-s-transferase in bacterial cells. Briefly, the EcoRI fragment described above was cloned into the EcoRI site of pGEX-4T-1 (Pharmacia). Expression of the fusion protein in E. coli was induced by addition of IPTG (1 mM) to the culture medium. After 4 hours of this regimen, cells were pelleted and resuspended in PBS plus various protease inhibitors. The cell suspension was then sonicated and centrifuged to pellet the cell debris. The soluble fraction was collected and analyzed on SDS-PAGE and tested for phosphatase activity. The expression of the fusion protein was confirmed by Western Blot using an anti-GST antibody. As demonstrated in FIGS. 1A and 1B, the recombinant Candida TYP1 phosphatase was active against both para-nitrophenylphospate and fluoroscein diphosphate.

### EXAMPLE 2

### Cloning of Candida albicans CKS1

In similar fashion to the cloning of the Candida TYP1 gene, a suc1 homolog was cloned from a Candida genomic library by PCR amplification using the primers TWYGARTAYMGNCAYGTNATG and AANARNARDATRTGNGGYTC. As above, the PCR fractions were separated on an agarose gel, the fragment eluted, and cloned into pCRII. DNA probes were generated as ³²P-labeled nick translation products, and used to further screen a C. albicans cDNA library. Larger cDNA clones isolated by this technique were sequenced. The nucleotide sequence for the CKS1 open reading frame, plus flanking noncoding sequence, is provided in SEQ. ID. No. 2.

The CKS1 coding sequence was subcloned into a pQE vector (Qiagen), and used to produce native proteins. The purified proteins should isolate the Candida CDK1 from cell lysates.

### EXAMPLE 3

### Cloning of a Candida Cyclin-Dependent Kinase

Using the degenerate oligonucleotides TCNGGNGCNCKRTACCANARNGT and GGNGARGGNACNTAYGGNGTNGT, a cyclin-dependent kinase was isolated from a C. albicans genomic library by PCR. The amplification program consisted of 30 cycles: 94° C. for 1 min., 50° C. for 1 min., and 72° C. for 1 min. Two major PCR reaction products were generated, separated on an agarose gel, and subsequently cloned into the pCRII vector, followed by standard Sanger sequencing. One of the two clones, a 490 bp fragment, exhibited a reasonable degree of similarity with other members of the CDK gene product family and was accordingly used to screen a C. albicans cDNA library.

Purified probes were generated as ³²P-labeled nick translation products, and hybridization was performed at 53° C. overnight in Church's solution (7% SDS, 250 mM NaP pH 7's, 10, mM, EDTA, pH 7) and filters were washed twice at the same temperature in a buffer obtaining 2×SSC and 0.1% SDS. The open reading frame for the cyclin-dependent kinase, referred to herein as CDK1, is given in SEQ. ID. No. 3.

### EXAMPLE 4

### Cloning of a C. albicans Cyclin

As above, the degenerate primers GANGANYKNGMNGANCCNYTNATG and ATNCKNCKNARRAARTTCAT were used to amplify C. albicans genomic DNA. The amplification program consisted of 30 cycles: 94° C. for 1 minute, 43° C. for 1 minute, and 72° C. for 1 minute. Two reaction products of about 450 and 700 bp were separated on an agarose gel. The 450 bp DNA fragment was reamplified and cloned into the pCRII vector and then used to screen a C. albicans cDNA library. An apparent cyclin B homolog, referred to herein as CYB1, was isolated from the cDNA library. The open reading frame for this cyclin is given by SEQ. ID. No. 4.

In C. albicans and C. maltosa, the CUG codon, which encodes leucine in the universal codon usage, is believed to be translated as serine (amino acid residues 301 and 383 of SEQ ID NO. 4). See, for example, Sugiyama et al. (1995) Yeast 11:43-52 and Zimmer et al. (1995) Yeast 11:33-41. Accordingly, it will be understood that an equivalent gene for expression in other cells can be modified at these positions to a codon for serine. However, it is noted that expression of the CYB1 gene in S. pombe produced what is apparently a functional protein, suggesting that these residues do not effect the biological activity of the cyclin, or that Sugiyama et al. were incorrect.

Sequence CLUSTAL alignment method (Higgins et al. (1992) Comp. Appl. Bio-Sci. 8:189-191) was run on the MegAlign program in the DNAStar package showed that the C. albicans CYB1 gene product is 34.8%, 34.4%, 35.5%, 33.3%, and 33.7% identical to the S. cerevisiae Clb1, Clb2 (Fitch et al. (1992) Mol. Biol. Cell 3:805-818), S. pombe Cdc13 (Booher et al.(1988) EMBO J. 7:2321-2327; Hagan et al. (1988) J. Cell Sci. 91:587-595), Cig2 (Connolly et al. (1994) Mol. Cell. Biol 14:768-776) and A. nidulans NimE (O'Connell et al. (1992) EMBO J 11:2130-2149) proteins, respectively. Percentages of identity increase up to 57% when only the C-terminal parts, containing the cyclin box, of the fungi B-type cyclins are aligned. The destruction box (RQYLGDVSN, amino acids 67 to 75 of CYB1) matches perfectly the consensus RxxLxxxxN which is essential for cyclin degradation via the ubiquitin pathway (Glotzer et al. (1991) Nature 349:132-138). The P box, which is required for Cdc25 activation by the MPF complex (Galaktionov et al. (1991) Cell 67:1181-1194; Zheng et al. (1993) Cell 75:155-164) is also present on the C. albicans Cyb1 protein (amino acids 237 to 268, SEQ ID NO. 4). Cyb1 P box is 58.8%, 64.7%, 67.6%, 61.8% and 70.6% identical to the S. cerevisiae Clb1, Clb2, S. pombe Cdc13, Cig2, and A. nidulans NimE P boxes, respectively.

### EXAMPLE 5

### C. albicans CDK1 Complements the S. pombe cdc2-33 Temperature Sensitive Mutation.

To test if the CDK1 cDNA is a functional gene the full length CDK1 cDNA was cloned into the S. pombe pART1 expression vector (McLeod et al. (1987) EMBO J. 6:729-736), yielding pCDK1.5. pART1 contains the S. cerevisiae LEU2 gene that complements a S. pombe leu1-32 mutation, the S. pombe ars1 sequence, and the S. pombe adh promoter which initiates strong and constitutive transcription. pCDK1.5 was used to transform the temperature sensitive S. pombe cdc2-33 strain (Nurse et al. (1976) Nature 146:167-178). Transformants were obtained at 25° C., which is the permissive temperature for cdc2-33. They were then streaked for single colonies and incubated at 25° C., 35° C., or 37° C. The C. albicans CDK1 gene enables a S. pombe cdc2-33 strain to form colonies at both 35° C. and 37° C., however, complementation is not as good as when the S. pombe wild-type cdc2 gene is used.

### EXAMPLE 6

### C. albicans CYB1 Complements the S. pombe cdc12-117 Temperature Sensitive Mutation.

To test if the CYB1 cDNA is a functional gene the full length CYB1 cDNA was cloned into the S. pombe pART1 expression vector (Mc Leod et al. (1987) EMBO J. 6:729-736). The resulting plasmid pCYB1.5 was used to transform a temperature sensitive S. pombe cdc13-117 strain (Nasmyth et al. (1981) Mol. Gen. Genet. 182:119-124). Transformants obtained at 25° C. were then streaked for single colonies and incubated at 25° C., 35° C., or 37° C. The C. albicans CYB1 gene product is able to rescue a S. pombe cdc13-117 mutation at 35° C., but no colony formation was observed at 37° C., indicating a partial rescue.

### EXAMPLE 7

### Interaction Between the CDK1 and CYB1 Proteins

Using the primers GACCAACACGAATTCCAAATGGTAGAGTTATCTG and TGAGGAGTCGACCAAGATTTATTGCATG, which contain EcoRI and a SalI restriction sites, respectively, the CDK1 coding sequence was amplified and subcloned into pEG202 vector in order to created a CDK1-LexA fusion protein. Likewise, the CYB1 coding sequence was amplified with the oligonucleotides CATTTTGAATTCATAGTAATGCCACAAGTC and ATAGTCCTCGAGACTTTACTCTTCTGCTTC, cut with EcoRI and XhoI, and the restriction fragment was subcloned into the vector pJG4-5 (Gyuris et al. (1993) Cell 75:791-803) in order to generate a CYB1-VP16 fusion protein.

The two vectors were used to simultaneously transform the S. cerevisiae strain YEG048 so as to constitute an interaction trap assay. Analysis of the transformants revealed that the CDK1 and CYB1 proteins interact with one another.

### EXAMPLE 8

### Generation of a TYP1-Dependent Hypermitotic Cell

When the TYP1 plasmid construct pART-TYP1, described above, is used to transform the S. Pombe strain Sp553 (h+N, cdc25-22, wee1-50, leu1-32) using well known procedures. Briefly, cells are grown in YE medium at 25° C. until they were in exponential phase (˜10⁷ cells/ml). The cells are then spun down from the media at 3000 rpm for 5 minutes, and resuspended in LiCI/TE at a concentration of ˜10⁸ cells/ml (LiCl/TE=10 mM Tris, 1 mM EDTA, 50 mM LiCI, pH 8). The resuspended cells are incubated at room temperature for 10 minutes, then spun again at 300 rpm for 5 minutes, resuspended in LiCI/TE to a concentration of ˜5×10⁸ cells/ml, and shaken for 30 minutes at 25° C.

To an aliquot of 1501 μl of cells, 500 ng of plasmid DNA and 350 μL of PEG/TE (10 mM Tris, 1 mM EDTA, 50% PEG 4000, pH 8) is added. The cell/plasmid mixture is then incubated for 30 minutes at 25° C., heat shocked at 42° C. for 20 minutes, then spun at 15,000 rpm for 10 seconds after the addition of 0.5 mL of Edinburgh Minimal Medium (EMM). The cells were resuspended in 0.6 mL EMM, and 0.2 mL aliquots were plated.

At the non-permissive temperature of 37° C., both the endogenous wee1 and CDC25 activities of the Sp553 cells are impaired such that they mutually off-set each other's effects, and the cells are still able to proliferate. However, the effect of expressing the recombinant Candida TYP1 protein in a yeast “wee” background results in mitotic catastrophe. For example, at the permissive temperature of 25° C. (wee1 is expressed) the cells are able to proliferate. However, shifting the temperature to the non-permissive temperature of 37° C. results in mitotic catastrophe.

### EXAMPLE 9

### Assay for TYP1 Inhibitors Using a Hypermitotic Cell

To assay the anti-mitotic activity of various candidate agents, the cells of Example 6 are either plated on a solid medium such as EMM plates or suspended in an appropriate vegetative broth such as YE.

In the instance of plating on a solid medium, candidate agents are subsequently blotted onto the plate, and the plate incubated at the non-permissive temperature of 37° C. A halo of cell growth will form surrounding those agents able to at least partially inhibit a mitotic activator which can rescue the otherwise catastrophic cell.

Where growth of the cells is carried out in a vegetative broth, aliquots of cell/media are placed in the wells of microtitre plates and serial dilutions of candidate agents are added to the wells. The plates are incubated at 37° C., and the A₅₉₅ for each well measured over time and compared to similar wells of cells/media which lack the candidate agent (e.g. negative controls). An increase in absorbence over time relative to the negative controls indicates positive proliferation of the cells and suggests an ability of a particular candidate agent to inhibit a mitotic activator.

All of the above-cited references and publications are hereby incorporated by reference.

### Equivalents

Those skilled in the art will recognize, or be able to ascertain using no more than routine experimentation, numerous equivalents to the specific assay and reagents described herein. Such equivalents are considered to be within the scope of this invention and are covered by the following claims.

1. A substantially pure preparation of a Candida CAK1 polypeptide.

2. The CAK1 polypeptide of claim 1, wherein the CAK1 polypeptide comprises an amino acid sequence at least 75 percent homologous to an amino acid sequence represented in SEQ ID No. 14.

3. The CAK1 polypeptide of claim 1, which polypeptide functions in one of either role of an agonist or an antagonist of cell cycle regulation of a Candida cell.

4. The CAK1 polypeptide of claim 1, which polypeptide has an intrinsic kinase activity.

5. The CAK1 polypeptide of claim 4, wherein the kinase activity of the CAK1 polypeptide activates a Candida cyclin dependent kinase.

6. An immunogen comprising the polypeptide of claim 1, in an immunogenic preparation, said immunogen being capable of eliciting an immune response specific for the Candida CAK1 polypeptide.

7. An antibody preparation specifically reactive with the polypeptide of claim 1.

8. A recombinantly produced Candida CAK1 polypeptide.

9. The CAK1 polypeptide of claim 8, having an amino acid sequence at least 75 percent homologous to an amino acid sequence designated by SEQ ID No. 14.

10. The CAK1 polypeptide of claim 8, which polypeptide is a protein kinase.

11. The CAK1 polypeptide of claim 8, which polypeptide is a fusion protein.

12. The CAK1 polypeptide of claim 8, which polypeptide phosphorylates Candida cyclin dependent kinases (cdks).

13. The CAK1 polypeptide of claim 8, which polypeptide binds to a Candida cyclin-dependent kinase.

14. A substantially pure nucleic acid comprising a nucleotide sequence which encodes a CAK1 polypeptide at least 75% homologous to an amino acid sequence represented in SEQ ID No. 14.

15. The nucleic acid of claim 14, wherein the encoded CAK1 polypeptide functions in one of either role of an agonist or an antagonist of cell cycle regulation of a Candida cell.

16. The nucleic acid of claim 14, wherein the encoded CAK1 polypeptide has an intrinsic kinase activity.

17. The nucleic acid of claim 16, wherein the kinase activity of the CAK1 polypeptide activates a Candida cyclin dependent kinase.

18. The nucleic acid of claim 16, wherein the phosphatase activity of the CAK1 polypeptide phosphorylates Candida cyclin dependent kinases (cdks).

19. The nucleic acid of claim 14, which nucleic acid further comprises a transcriptional regulatory sequence operably linked to said nucleotide sequence so as to render said nucleotide sequence suitable for use as an expression vector.

20. An expression vector, capable of replicating in at least one of a prokaryotic cell and eukaryotic cell, comprising the nucleic acid of claim 14.

21. A host cell transfected with the expression vector of claim 20.

22. A method of producing a recombinant Candida CAK1 protein comprising culturing the cell of claim 21 in a cell culture medium to express said CAK1 protein and isolating said CAK1 protein from said cell culture.

23. A probe/primer for identifying nucleic acid encoding a regulatory protein of a Candida cell, which probe/primer comprises a nucleotide sequence which hybridizes under stringent conditions to at least 10 consecutive nucleotides of sense or antisense sequence of a nucleic acid selected from a group consisting of SEQ ID No. 1, SEQ ID No. 2, SEQ ID No. 3, SEQ ID No. 4, SEQ ID No. 5, SEQ ID No. 6 and SEQ ID No. 13.

24. The probe/primer of claim 23, further comprising a label group attached thereto and able to be detected.

25. A diagnostic test kit for identifying nucleic acid of a Candida organism, comprising the probe/primer of claim 23, for measuring a level of a nucleic acid encoding the regulatory protein in a biological sample.

26. A method of identifying a compound which is an inhibitor of CAK1 kinase, comprising the steps of:
a) generating a combination including:
1) a test agent to be assessed;
2) a cell free preparation of a CAK1 kinase from Candida, and
3) a substrate of the CAK1 kinase, other than an active cyclin dependent kinase (CDK);
b) maintaining the combination under conditions appropriate for the CAK1 kinase to convert the substrate to product; and
c) measuring the conversion of the substrate to product,
wherein a statistically significant decrease in the conversion of substrate to product in the combination, relative to a control comprising CAK1 kinase and the substrate and lacking the test agent, indicates that the test compound is an inhibitor of the CAK1 kinase.

27. The method of claim 26, wherein the CAK1 kinase is a component of a fusion protein.

28. The method of claim 27, wherein the fusion protein is a glutathione-S-transferase/CAK1 kinase fusion protein.

29. The method of claim 26, wherein the conversion of substrate to product provides a calorimetric indicator of kinase activity.

30. The method of claim 29, wherein the substrate is a synthetic substrate of CAK1 kinase comprising a calorimetric label which is detectable when the substrate is converted to product.

31. The method of claim 26, wherein the CAK1 kinase comprises a polypeptide having an amino acid sequence represented in SEQ ID No. 14.

32. An assay for screening test agents for an inhibitor of an interaction of a cyclin dependent kinase (CDK) with a CAK1 polypeptide, comprising:
i) generating a combination including:
a) a test agent to be assessed;
b) a cell free preparation of a CAK1 polypeptide from Candida; and
c) a cyclin dependent kinase;
ii) detecting the formation of a complex including said CDK and said CAK1 polypeptide,
wherein a statistically significant decrease in the formation of said complex in the presence of said test agent, relative to the formation of a CDK/CAK1 complex in the absence of said test agent, is indicative of said test agent being an inhibitor of the interaction between said CDK and said CAK1 polypeptide.

33. A method for screening test agents for an inhibitor of an interaction of a cyclin dependent kinase (CDK) with a CAK1 polypeptide, comprising:
i) providing an interaction trap system including
a) a first fusion protein comprising a cyclin-dependent kinase (CDK) portion, and
b) a second fusion protein comprising a Candida CAK1 protein portion,
c) maintaining the interaction trap system under conditions wherein said interaction trap system is sensitive to interactions between the CDK portion of said first fusion protein and said CAK1 protein portion of said second polypeptide;
ii) contacting said interaction trap assay with a test agent;
iii) measuring the interactions between said fusion proteins in the presence of said candidate agent; and
iv) comparing the interactions of said fusion proteins in the presence of said candidate agent to interactions of said fusion proteins in the absence of the candidate agent,
wherein a statistically significant decrease in the level of interaction of the fusion proteins in the presence of said candidate agent is indicative of the test agent being an inhibitor of interactions between CDK and the CAK1 protein.

34. An assay for identifying an inhibitor of a pathogen CAK1 kinase, comprising
i. providing a cell expressing a recombinant CAK1 kinase from Candida, said cell having an impaired checkpoint which causes premature entry of the cell into mitosis resulting in cell death, the premature entry into mitosis being mediated at least in part by the CAK1 kinase;
ii. contacting the cell with a candidate agent;
iii. measuring a level of proliferation of the cell in the presence of the candidate agent; and
iv. comparing the level of proliferation of the cell in the presence of the candidate agent to a level of proliferation of the cell in the absence of the candidate agent,
wherein a statistically significant increase in the level of proliferation in the presence of the candidate agent is indicative of inhibition of the CAK1 kinase by the candidate agent.

35. The assay of claim 34, wherein the cell-cycle checkpoint impairment comprises a increase in CAK1 activating phosphorylation of a cyclin-dependent kinase (CDK).

36. A Schizosaccharomyces cell comprising an expressible recombinant gene encoding an exogenous CAK1 kinase from Candida.
